(19) United States
(12) Patent Application Publication
(10) Pub. No.: US 2026/0091158 A1
(43) Pub. Date: Apr. 2, 2026

(54) NON-PYROGENIC BACTERIAL CELLULOSE FOR BIOMEDICAL APPLICATIONS AND METHOD OF MANUFACTURE THEREOF

(71) Applicant: Laboratoires AxCell, Sherbrooke (CA)

(72) Inventors: Jérémie Chaussé, Sherbrooke (CA); Vincent-Daniel Girard, Sherbrooke (CA)

(21) Appl. No.: 19/375,133

(22) Filed: Oct. 30, 2025

Related U.S. Application Data
(63) Continuation of application No. PCT/CA2025/051255, filed on Sep. 24, 2025.

(30) Foreign Application Priority Data
Oct. 2, 2024 (GB) 2414484.2

(51) Int. Cl. A61L 27/20; A61L 26/00; A61L 27/52; A61L 27/56; C08L 1/02

(52) U.S. Cl. CPC A61L 27/20; A61L 26/0023; A61L 26/008; A61L 26/0085; A61L 27/52; A61L 27/56; C08L 1/02; A61L 2430/34

(57) ABSTRACT

The disclosure relates to biomaterials, specifically non-pyrogenic bacterial cellulose for implantable medical applications, and to a depyrogenation process. It addresses the problem that sterile bacterial cellulose remains pyrogenic due to endotoxin, particularly lipopolysaccharide, which resists standard sterilization and purification. The process involves providing a purified bacterial cellulose membrane in an aqueous medium and subjects it to depyrogenation at about 140° C.-250° C. under pressure sufficient to maintain the aqueous phase liquid. The treated material is a bacterial cellulose hydrogel having a water holding capacity of at least 100 g water/g dry bacterial cellulose and endotoxin below 0.5 EU/mL as determined by a Limulus amebocyte lysate assay on enzymatically degraded samples. The product and process are useful for manufacturing non-pyrogenic implantable bacterial cellulose devices, including internal implants, tissue scaffolds, and other biocompatible medical components.

### CROSS-REFERENCE TO RELATED APPLICATION

The present application claims the benefit of Great Britain patent application serial number 2414484.2 filed on Oct. 2, 2024. The contents of the above-referenced document are incorporated herein by reference in their entirety.

### TECHNICAL FIELD

This application generally relates to the field of soft tissue implants and, more specifically, to non-pyrogenic bacterial cellulose for biomedical applications and method of manufacturing and using same.

### COPYRIGHT

A portion of the disclosure of this patent document contains material that is subject to copyright protection. The copyright owner has no objection to the facsimile reproduction by anyone of the patent document or the patent disclosure as it appears in the Patent and Trademark Office patent file or records, but otherwise reserves all copyright rights whatsoever.

### BACKGROUND

Soft tissue implants play a crucial role in reconstructive surgery. These procedures aim to restore, repair, enhance, or promote the health of the body's native soft tissues. Soft tissue grafts are also important in the health sector. Among other things, they help in tissue regeneration, promote healing, and reduce the risk of complications in high-risk procedures.

Several soft tissue implants are available on the market today. Collagen-based implants, such as AlloDerm® (BioHorizon, Birmingham, AL, USA), face significant challenges in terms of cost, largely due to the use of autologous cell culture to enhance biocompatibility. This method, while effective, renders these implants expensive. Additionally, many bovine and porcine implants, including ZYDERM®, ZYPLAST®, and CosmoDerm® (all formerly produced by Inamed Corporation, Santa Barbara, CA, USA), were withdrawn from the U.S. market due to their reactivity. The high costs associated with modern resorbable membranes have subsequently driven interest in alternative, cost-effective materials for use as scaffolds in tissue engineering and as fillers.

Among these alternative biopolymers, materials such as chitosan, silk, alginate, and starch have garnered attention. However, one of the most promising naturally sourced polymers for biomedical applications is cellulose, particularly in its nanocrystalline form. Nanocrystalline cellulose is highly valued in the medical field due to its purity, excellent mechanical properties, antibacterial characteristics, and large surface area.

Nanocrystalline cellulose can be derived either from plant sources or produced by bacteria. While plant-derived cellulose is widely used in industrial applications, the necessity to remove lignin and hemicellulose increases production costs and can detract from the material's final properties. Moreover, the inherent properties of plant-derived nanocrystalline cellulose are relatively unsuitable for medical applications, primarily due to the challenges associated with converting it into a hydrogel.

Bacterial cellulose, on the other hand, is frequently the focus of research into biomedical polymers. Bacterial cellulose is naturally produced in a nanocrystalline form through fermentation and requires only minimal purification steps to remove organic matter, making it a more viable candidate for medical use. Bacterial cellulose has many beneficial properties, such as high tensile strength, excellent water holding capacity, which, in turn, spark an interest in bacterial cellulose as a biomedical material of natural origin (Pogorelova et al., (2020). Bacterial Cellulose Nanocomposites: Morphology and Mechanical Properties. Materials 13, 2849).

Unmodified, its high-water content, purity, malleability, non-toxicity, and tensile strength make it very desirable when used as a hydrated wound dressing or as cartilage. Since early 2000, commercially sold products for wound treatments have become available, namely Dermafill® (Fibrocel Produtos Biotechnologies LTDA, Brazil), Bionext® (Bennett Health, Inc, Southlake, TX, USA), Membracel® (Vuelo Pharma, Curitiba/PR, Brasil), and Xcell® (Xylos Corporation, Langhorne, PA, USA). As these products are easier to get approved by the FDA (or other regulatory health organizations), it is unsurprising that the first medical products made from bacterial cellulose are external, unmodified (or infused with antibacterial agents), and non-permanent.

A critical requirement for implantable bacterial cellulose products, as opposed to bacterial cellulose membranes used for external applications, is that they must be non-pyrogenic. This requirement becomes increasingly important as the next generation of bacterial cellulose products moves towards internal implantation. The pyrogenicity of sterile bacterial cellulose products is primarily due to the presence of endotoxins, with lipopolysaccharides (LPS) being the most significant contributor. These compounds are particularly challenging to remove because they are resistant to standard sterilization and purification procedures. Addressing the issue of pyrogenicity is crucial for the successful development and deployment of implantable bacterial cellulose products in medical applications.

In light of the above, there remains a need to provide improved soft tissue implants that address at least some of the aforementioned disadvantages.

### SUMMARY

This Summary is provided to introduce a selection of concepts in a simplified form that are further described below in the Detailed Description. This Summary is not intended to identify key aspects or essential aspects of the claimed subject matter.

As embodied and broadly described herein, the present disclosure relates to a non-pyrogenic bacterial cellulose characterized as being a hydrogel having a water holding capacity of at least 100 g water/g dry bacterial cellulose and having less than 0.5 EU/mL of endotoxins, measured with a Limulus amebocyte lysate test on a sample of the bacterial cellulose membrane which has been enzymatically degraded.

As embodied and broadly described herein, the present disclosure relates to a non-pyrogenic bacterial cellulose characterized as being a hydrogel having a water content of at least 95 wt. % and having less than 0.5 EU/mL of endotoxins, measured with a Limulus amebocyte lysate test on a sample of the bacterial cellulose membrane which has been enzymatically degraded.

In some embodiments, the non-pyrogenic bacterial cellulose includes one or more of the following features:
the water holding capacity is of at least 100 g water/g dry bacterial cellulose, at least 150 g water/g dry bacterial cellulose, or at least 200 g water/g dry.
the water content is of at least 96 wt. %, at least 97 wt. %, or at least 98 wt. %, or at least 99 wt. %.
the hydrogel is in a membrane form.
the membrane has a thickness of from about 2 mm to about 20 mm.
it is characterized with an average cell penetration of from about 7 μm/day to about 15 μm/day.
It comprises pores having a size facilitating cell migration and neovascularization.
It is for use in drug delivery, tissue engineering, soft tissue reconstruction or replacement, or regenerative medicine.

As embodied and broadly described herein, the present disclosure relates to a manufacturing method, comprising: providing a purified bacterial cellulose membrane in an aqueous solution; performing a depyrogenation process under conditions sufficient to obtain an endotoxin level of less than 0.5 EU/mL measured with a Limulus amebocyte lysate test on a sample of the bacterial cellulose membrane which has been enzymatically degraded, wherein the conditions include a process temperature within the range of from about 140° C. to about 250° C. and a pressure which allows the aqueous solution to stay liquid.

In some embodiments, the method includes one or more of the following features:
the pressure is equal to the saturated vapor pressure of water at the process temperature.
the pressure is within the range of from about 0.36 MPa at 140° C. to about 4 MPa at 250° C.
the depyrogenation process is performed for a period of from about 30 minutes to about 3 hours.
the depyrogenation process is performed in a high-pressure, high-temperature autoclave unit.
It further comprises cooling the bacterial cellulose membrane to room temperature.
It further comprises packaging the bacterial cellulose membrane in a sterile, non-pyrogenic, and hydrated pouch.
the aqueous solution is isotonic saline solution or deionized water.

All features of exemplary embodiments which are described in this disclosure and are not mutually exclusive can be combined with one another. Elements of one embodiment can be utilized in the other embodiments without further mention. Other aspects and features of the present invention will become apparent to those ordinarily skilled in the art upon review of the following description of specific embodiments in conjunction with the accompanying Figures.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

A detailed description of specific exemplary embodiments is provided herein below with reference to the accompanying drawings in which:

FIG. 1 is a non-limiting illustration of pyrogenic substances released upon gram-negative bacteria cell lysis.

FIG. 2A to FIG. 2D show native bacterial cellulose used on a diabetic rat as a wound dressing. The pictures are in chronological order (A) day 0, (B) day 3, (C) day 10, and (D) day 14.

FIG. 2E shows a non-limiting histological picture of purified bacterial cellulose (purified with hot treatment), 12 weeks after implantation in a healthy rat.

FIG. 3 is a non-limiting picture of a non-pyrogenic bacterial cellulose membrane, in accordance with embodiments of the present disclosure.

FIG. 4A and FIG. 4B are scanning electron microscopy (SEM) images of native bacterial cellulose and non-pyrogenic bacterial cellulose according to some embodiments of the present disclosure, respectively.

FIG. 5A and FIG. 5B show non-limiting histological pictures of a medical grade silicone implant (12 weeks after implantation in a rat's hypodermis) and non-pyrogenic bacterial cellulose in accordance with embodiments of the present disclosure (2 weeks after implantation in a rat's hypodermis), respectively.

FIG. 5C and FIG. 5D show non-limiting histological pictures of hot purified bacterial cellulose (12 weeks after implantation in a rat's hypodermis) and medical grade silicone implant (12 weeks after implantation in a rat's hypodermis), respectively.

FIG. 6A and FIG. 6B show non-limiting histological pictures of a pyrogenic bacterial cellulose implant (12 weeks after implantation in a rat's dermis) and non-pyrogenic bacterial cellulose in accordance with embodiments of the present disclosure (2 weeks after implantation in a rat's dermis), respectively.

FIG. 7 is a non-limiting flowchart of a method for producing non-pyrogenic bacterial cellulose, in accordance with some embodiments of the present disclosure.

FIG. 8 is a non-limiting flowchart of exemplary steps of the method of FIG. 7, in accordance with some embodiments of the present disclosure.

FIG. 9 is a non-limiting flowchart of exemplary steps of the method of FIG. 7, in accordance with some embodiments of the present disclosure.

FIG. 10 is a non-limiting histogram showing the water content (%) in various bacterial cellulose.

FIG. 11 is a non-limiting histogram showing the water holding capacity (A. U.) in various bacterial cellulose.

FIG. 12 is a non-limiting picture showing locations where cell penetration has been measured in a bacterial cellulose of the present invention.

FIG. 13 is a non-limiting graph of various measurements made on the bacterial cellulose of FIG. 12, in accordance with some embodiments of the present disclosure.

In the drawings, exemplary embodiments are illustrated by way of example. It is to be expressly understood that the description and drawings are only for the purpose of illustrating certain embodiments and are an aid for understanding. They are not intended to be a definition of the limits of the invention.

### DETAILED DESCRIPTION

The present technology is explained in greater detail below. This description is not intended to be a detailed catalog of all the different ways in which the technology may be implemented, or all the features that may be added to the instant technology. For example, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art considering the instant disclosure which variations and additions do not depart from the present technology. Hence, the following description is intended to illustrate some embodiments of the technology, and not to exhaustively specify all permutations, combinations, and variations thereof.

### Comparative Soft-Tissue Replacement Materials

The following discussion provides a systematic overview of representative materials presently employed, or proposed for use, in soft-tissue augmentation and reconstruction. For each material, the salient attributes (namely intrinsic hydration capacity, attainable fill volume, anticipated host inflammatory response, propensity for cellular integration, expected in vivo lifetime, clinical accessibility, commercially available physical forms, indicative cost profile, and principal limitations) are set forth to facilitate an informed evaluation of their suitability for implantation relative to the presently disclosed invention.

### 1. Autologous Fat

Autologous adipose tissue exhibits inherently high water content, thereby contributing favorable hydration that supports local tissue regeneration. Typical graft volumes range from low to medium, limited principally by harvestable donor fat reserves and the need to preserve vascular integrity at the recipient site. Owing to its autologous origin, the inflammatory response is negligible; nevertheless, an initial resorptive phase leads to approximately 30-50% volumetric loss within the first post-operative months. Adipocytes and associated stromal vascular fraction integrate effectively with host tissue, rendering the surviving fraction essentially permanent. Accessibility is contingent on the patient possessing adequate donor deposits, and procurement requires liposuction, centrifugation, and reinjection, collectively increasing operative complexity. Costs are procedure-dependent and generally elevated relative to off-the-shelf fillers. Drawbacks include donor-site morbidity, variable graft take, and the equipment burden (e.g., centrifuge) necessary for processing.

### 2. Bacterial Cellulose

Bacterial cellulose (BC), while already available in certain wound-dressing formats, has limited application in bulk soft-tissue replacement due to its intrinsically low hydration and lack of injectable preparations. Implanted BC demonstrates minimal acute inflammation and acceptable biocompatibility, with cellular integration characterized by surface contact rather than deep cellular infiltration. Material persistence is effectively permanent under physiological conditions owing to the absence of human cellulase enzymes. Presently, BC suitable for large-volume soft-tissue filling is not commercially accessible, although solid implantable forms may be fabricated. Pricing is moderate; however, manufacturing processes must mitigate potential pyrogenicity to avoid febrile reactions. The principal disadvantages stem from its rigidity relative to native tissue and the aforementioned pyrogenic risk upon implantation.

### 3. Bovine Collagen

Bovine-derived collagen possesses high intrinsic hydration, affording a pliable matrix conducive to soft-tissue contouring. Usable volumes are typically low, reflecting the reconstituted gel's limited structural stability. Clinical reports indicate up to 3% hypersensitivity incidence, which historically prompted multiple market withdrawals between 1990 and 2010. The collagen scaffold permits extensive cell migration and integration; however, enzymatic degradation restricts in vivo durability to roughly 12 months. Material is readily obtainable in both implantable sheets and prefilled syringes. Costs are moderate, although recombinant or non-bovine alternatives command higher pricing. Drawbacks include potential antigenicity, transient effect, and religious or dietary concerns associated with porcine or bovine origins.

4. Expanded Polytetrafluoroethylene (ePTFE)

ePTFE exhibits minimal water uptake, conferring extremely low hydration. Manufactured as porous solid sheets or rods, it accommodates a wide range of implant volumes. Acute inflammation is negligible; yet, progressive fibrous encapsulation commonly occurs, reducing long-term integration. Material longevity is essentially permanent but reversible via explantation. Because ePTFE implants are pre-formed, clinical accessibility is high. The cost is categorized as high, reflecting specialized fabrication and sterilization. A key disadvantage is the potential for palpability and unnatural contouring when placed within mobile subdermal planes.

### 5. Hyaluronic Acid (HA)

Hyaluronic acid is a highly hydrophilic polysaccharide that furnishes substantial tissue hydration upon implantation. Commercial cross-linked gels are optimized for low-volume facial applications. Immunogenicity is negligible, and foreign-body inflammation is minimal. The bioresorbable matrix supports only limited cellular integration before enzymatic depolymerization, yielding a functional lifetime of up to 12 months. HA fillers are widely accessible in preloaded syringes, permitting rapid office-based administration. Pricing is uniformly high on a per-milliliter basis. Limitations include the temporary nature of the augmentation and the potential for Tyndall effect or migration in poorly vascularized regions.

### 6. Calcium Hydroxyapatite

Microspheres of calcium hydroxyapatite (CaHA) suspended within an aqueous carrier deliver moderate hydration and allow low-to-medium augmentative volumes. The material elicits low inflammatory response, although nodular formation has been reported, particularly following lip injections. Cellular integration is moderate, primarily involving fibrous encapsulation around individual particles. Clinical durability extends to approximately 18 months, after which gradual resorption occurs. CaHA formulations are readily available for injection and can also be incorporated into implantable constructs. Product costs are high, and usage generally necessitates an additional carrier gel for proper dispersion. Applications are preferentially directed toward periosteal or osseous mimetic sites due to the material's radiopacity and mechanical stiffness.

### 7. Poly-L-Lactic Acid (PLLA)

PLLA microparticles provide a non-hydrating scaffold that induces neocollagenesis over time. Injectable suspensions accommodate low-to-medium volumetric correction. Initial inflammatory response is minimal; however, controlled foreign-body reaction is requisite for therapeutic efficacy. Integration is moderate and mediated by de novo collagen deposition rather than direct incorporation. Bioavailability persists for up to two years, with gradual hydrolytic breakdown into lactic acid monomers. Devices and injectables are widely distributed, contributing to high accessibility; associated costs are comparatively high. The staged resorption process often necessitates multiple treatment sessions and may produce transient nodules if reconstitution or injection technique is suboptimal.

### 8. Polymethyl Methacrylate (PMMA)

PMMA is a non-hydrating, chemically inert polymer supplied as solid implants or as microspheres in a bovine collagen carrier. Injectable and implantable formats support a broad spectrum of volumes. Acute inflammation remains low, but long-term fibrous encapsulation predominates, and true cellular integration is absent. PMMA is considered permanent; however, the material is difficult to remove once implanted. Clinical accessibility is high given extensive manufacturing infrastructure. Pricing falls at the higher end of the spectrum, reflecting stringent quality control requirements. Reported drawbacks encompass risk of late infection, potential implant instability, and, in breast applications, occurrence of “red breast syndrome.”

### 9. Silicone Elastomer and Gel

Medical-grade silicone yields no intrinsic hydration and is typically fabricated into cohesive gel-filled or solid elastomer implants. Volume capacity ranges from small to large, enabling whole-organ reconstruction. The material provokes minimal acute inflammatory reaction, yet encapsulation and capsular contracture are recognized sequelae. Cellular integration is absent; the implant remains distinct from host tissue. Functional lifetime is indefinite, though device failure-manifesting as rupture or leakage-necessitates replacement. Silicone implants are widely accessible and priced at moderate levels. Long-term disadvantages include potential migration, difficulty in radiographic assessment, and the need for explantation upon mechanical compromise.

### SUMMARY

Collectively, the foregoing survey illustrates that no single existing material simultaneously provides high hydration, substantial volumetric stability, minimal immunogenicity, durable integration, convenient delivery, and predictable long-term performance. Each material presents distinct trade-offs between biocompatibility, mechanical behavior, longevity, and economic or procedural considerations. These limitations underscore the necessity for improved compositions and delivery methodologies, as further described in the ensuing sections of this specification.

### Non-Pyrogenic Bacterial Cellulose of the Present Disclosure

The present inventors have through R&D work surprisingly and unexpectedly developed non-pyrogenic bacterial cellulose for use as bio-integrable matrix for human tissue substitutes, closure reinforcement, suture buttressing, guided tissue regeneration, musculoskeletal applications, active agent delivery and tissue engineering scaffolds, preferably for use in drug delivery, tissue engineering, soft tissue reconstruction or replacement, or regenerative medicine. More preferably, for use as soft tissue implant.

The non-pyrogenic bacterial cellulose of the present disclosure is characterized with one or more advantageous technical characteristics. For example, the non-pyrogenic bacterial cellulose of the present disclosure is characterized as combining one or more of increased hydration (i.e., increase water content, increase water holding capacity, etc.) compared to known non-pyrogenic bacterial cellulose, biocompatibility, minimal inflammation, cellular integration, permanence, volume, and accessibility while remaining affordable. Indeed, the cost associated with production of the herein described bacterial cellulose is minimal given that it requires very limited equipment for manufacturing and inexpensive ingredients. These advantages will be further described in the present disclosure.

### Hydration

In some implementations, the non-pyrogenic bacterial cellulose of the present disclosure is characterized by a highly hydrated hydrogel state.

For example, the non-pyrogenic bacterial cellulose can be characterized by a water holding capacity of at least 100 g water/g dry bacterial cellulose, such as at least 150 g water/g dry bacterial cellulose, at least 200 g water/g dry bacterial cellulose, at least 250 g water/g dry bacterial cellulose, or at least 300 g water/g dry bacterial cellulose. For example, the non-pyrogenic bacterial cellulose can be characterized by a water holding capacity of from about 100 g water/g dry bacterial cellulose to about 325 g water/g dry bacterial cellulose, preferably from about 200 g water/g dry bacterial cellulose to about 325 g water/g dry bacterial cellulose.

For example, the non-pyrogenic bacterial cellulose can be characterized by a moisture content of at least about 95 wt. % moisture, such as at least about 96 wt. %, at least about 97 wt. %, at least about 98 wt. %, or at least about 99 wt. %.

For example, the non-pyrogenic bacterial cellulose can be characterized by a combination of the afore-mentioned water holding capacity and moisture content.

Such high hydration rate of the non-pyrogenic bacterial cellulose of the present disclosure provides a technical effect. For example, it can provide the necessary hydration for proper tissue regeneration and help in surgeries where skin flaps might be very dry (for example, after chemotherapy or radiotherapy treatments). This allows the non-pyrogenic bacterial cellulose of the present disclosure to be used as a filling or volumetric augmentation medical implant, an advantage that competitor-produced bacterial cellulose cannot replicate.

In contrast, it is noted that the non-pyrogenic bacterial cellulose alternative currently available on the market is not characterized with such high hydration rate owing to its depyrogenation method which effectively removes endotoxins but uses a lengthy depyrogenation process that impacts the product's three-dimensional structure (rendering difficult to obtain a thick membrane, such as at least 2 mm) and hydration rate capability (e.g., see the results in the examples section of this specification). As such, this currently available bacterial cellulose is not used as a soft tissue replacement.

### Biocompatibility and Minimal Inflammation

In some implementations, the non-pyrogenic bacterial cellulose of the present disclosure is characterized by biocompatibility and minimal inflammation.

From a biocompatibility perspective, an implantable medical material (i.e., tissue replacement material) must be free from endotoxins (i.e., non-pyrogenic), microorganisms and other possible contaminants that interfere with the healing process and cause harm to the recipient. Implantable bacterial cellulose is considered by regulatory authorities as an implantable medical device and as such, must be free from endotoxins.

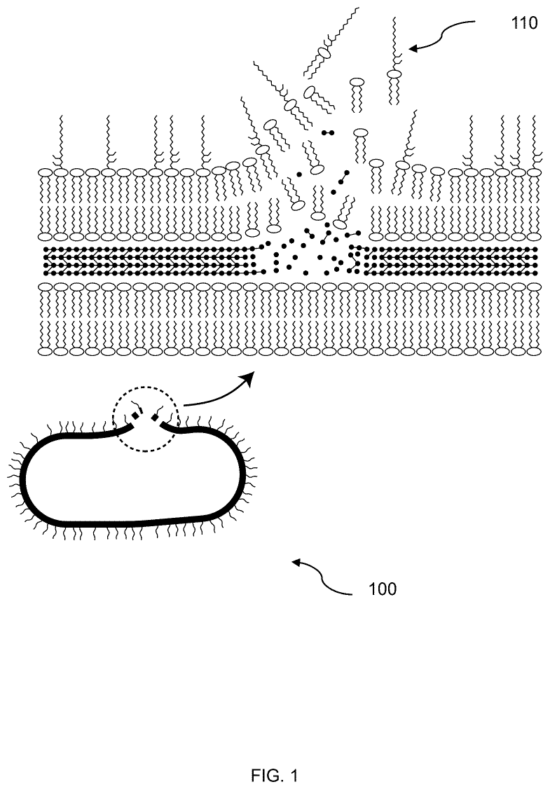

Because bacterial cellulose is produced from Gram-negative bacteria which naturally contain high levels of pyrogenic molecules such as lipopolysaccharides (LPS), bacterial cellulose is usually considered highly pyrogenic. With reference to FIG. 1, lysis of Gram-negative bacteria 100 causes a release of LPS 110 which “stick” tightly to the three-dimensional structure of the bacterial cellulose and is notoriously difficult to remove, as LPS are unaffected by standard sterilization and purification procedures (Schneier et al., Current technologies to endotoxin detection and removal for biopharmaceutical purification. Biotechnol Bioeng. 2020; 117(8):2588-2609. doi:10.1002/bit.27362; Gorbet et al., Endotoxin: The uninvited guest. Biomaterials. 2005; 26(34):6811-6817. doi:10.1016/j.biomaterials.2005.04.063; and Inselman et al., Bacterially derived medical devices: How commercialization of bacterial nanocellulose and other biofabricated products requires challenging of standard industrial practices. JBMR Part B. 2021; 109(11):1953-1959. doi:10.1002/jbm.b.34833).

Several approaches have been proposed to remove endotoxins from bacterial cellulose materials, which is called “depyrogenization” or “depyrogenation.” The principal methodologies identified to date may be grouped into: (i) iterative aqueous washing assisted by mechanical energy, (ii) supercritical fluid extraction, (iii) continuous alkaline perfusion, (iv) steam sterilization/autoclaving, (v) oxidative chemical treatment, (vi) thermal cycling, and (vii) combined alkali-solvent-adsorbent protocols. Each approach is summarized in Table 1.

TABLE 1

| | | | | Final | | | |
|---|---|---|---|---|---|---|---|
| | | | Tested | endotoxin | | | |
| Product | Method | Assay | component | concentration | | Concerns | Ref. |
| CNC in | Multiple | HEK | CNCs | Not fully | | | [1] |
| solution | ultrasonication | mTLR4 | solubilized at | specified | | | |
| | treatments | reporter | 10-100 | <5 EU/mL | | | |
| | and washes in | | ug/mL | | | | |
| | non-pyrogenic | | | | | | |
| | water. | | | | | | |
| Densified | Multiple | LAL test | Following | 27 | EU/mL | Tested | [2] |
| BC | supercritical | | ISO 10993- | | | extraction | |
| membranes | CO₂ | | 12 | | | media | |
| | treatments | | (76 h, 37° C.) | | | | |
| | and washes in | | | | | | |
| | non-pyrogenic | | | | | | |
| | water. | | | | | | |
| Densified | Sodium | PyroGene | Following | 0.1 | EU/mL | Tested | [3] |
| BC | hydroxide | recombinant | ISO 10993- | | | extraction | |
| membranes | perfusion | Factor | 12 | | | media | |
| | system and | C assay | (76 h, 37° C.) | | | | |
| | non-pyrogenic | | | | | | |
| | water washes | | | | | | |
| | over 42 days. | | | | | | |
| Bilayered | Sodium | PyroGene | Following | 0.15 | EU/mL | Tested | [4] |
| densified BC | hydroxide | recombinant | ISO 10993- | | | extraction | |
| membranes | perfusion | Factor | 12 | | | media | |
| | system and | C assay | (76 h, 37° C.) | | | | |
| | non-pyrogenic | | | | | | |
| | water washes | | | | | | |
| | over 42 days. | | | | | | |
| Porous BC | Unspecified | LAL test | Following | <0.1 | EU/mL | Unspecified | [5] |
| membranes | purification | | FDA | | | purification | |
| | method that | | Guidelines | | | method for | |
| | was also used | | on Validation | | | paraffin | |
| | to remove | | of LAL Test | | | removal | |
| | paraffin | | (1987) | | | Tested | |
| | microbeads, | | | | | extraction | |
| | followed by | | | | | media | |
| | autoclaving in | | | | | Use of | |
| | non-pyrogenic | | | | | withdrawn | |
| | water. | | | | | FDA | |
| | | | | | | Guidelines | |
| | | | | | | on Validation | |
| | | | | | | of LAL Test | |
| Oxidized BC | Sodium | Not | Not | Not | | Documentation | [6] |
| | hydroxide and | specified | specified | specified | | for treatment | |
| | hydrogen | | | | | with aqueous | |
| | peroxide | | | | | hydrogen | |
| | treatments. | | | | | peroxide | |
| | | | | | | scarce | |
| | | | | | | Unspecified | |
| | | | | | | tested | |
| | | | | | | components | |
| BC | Sodium | Not | Not | Not | | Documentation | [7] |
| membranes | hydroxide and | specified | specified | specified | | for treatment | |
| with | hydrogen | | | | | with aqueous | |
| polyhexamethylene | peroxide | | | | | hydrogen | |
| biguanide | treatments. | | | | | peroxide | |
| | | | | | | scarce | |
| | | | | | | Unspecified | |
| | | | | | | tested | |
| | | | | | | components | |
| BC | Sodium | LAL test | Not | <0.06 | EU/ml | Unspecified | [8] |
| membranes | hydroxide | | specified | | | tested | |
| | treatment. | | | | | component | |
| bacterial | Sodium | Not | Not | Not | | Unspecified | [9] |
| cellulose BC | hydroxide | specified | specified | specified | | tested | |
| membranes | treatment. | | | | | component | |
| BC | Series of | Not | Not | Not | | Unspecified | [10] |
| membranes | freeze- | specified | specified | specified | | tested | |
| | thawing. | | | | | component | |
| BC | Alternating | Not | Not | <8.84 | EU/mL | Unspecified | [11] |

| | | | | |
|---|---|---|---|---|
| membranes | sodium | specified | specified | tested |
| | hydroxide | | | component |
| | treatments, | | | |
| | ethanol | | | |
| | soaking, and | | | |
| | polysulfone | | | |
| | resin polymer | | | |
| | adsorption. | | | |

HEK: Human embryonic kidney cells,
LAL: Limulus amebocyte lysate,
CNCs: Cellulose nanocrystals,
BC: bacterial cellulose

The proposed approaches, however, do not demonstrate satisfying endotoxin levels from a regulatory and commercial perspective, or when tested in vivo. Without being bound by any theory, the present inventors believe that while several detection assays have been proposed to assess the efficiency of the proposed approaches, most detection assays involve a step of soaking the material in hot aqueous solution in an attempt to leach out endotoxins from the bacterial cellulose. Such leach out procedure, however, does not efficiently remove endotoxins from the woven fibers of bacterial cellulose due to their amphiphilic properties and “stickiness” to the three-dimensional structure of the bacterial cellulose, thus inducing false positive test when testing for the absence of endotoxins.

The present inventors believe that such problem with the endotoxin detection processes is not commonly recognized in the art.

The present inventors believe that variability in endotoxin removal efficiency from implantable bacterial cellulose may cause the scarcity of implantable bacterial cellulose products on the market. As of 2024, only one company provides an implantable medical product that includes bacterial cellulose, i.e., Syntecel Dura Repair™—which is partially dehydrated. The production process for this product requires many days to perform, as described in U.S. Pat. No. 8,198,261. This limits its application as a hydrogel for regenerative medicine or as a biocompatible bulking agent for plastic surgery.

### Cellular Integration

In some implementations, the non-pyrogenic bacterial cellulose of the present disclosure is characterized by an increased cellular integration rate.

Cellular integration is key for many soft tissue substitutes which translates into soft tissue material acceptance by the body. Acceptance by the body limits the risks of encapsulation that could potentially lead to the displacement of the medical implant. The non-pyrogenic bacterial cellulose of the present disclosure has a specific 3D structure which is absent from pyrogenic bacterial cellulose, as observed with a scan electronic microscope. The 3D structure is described in further details later in this text. The 3D structure of the non-pyrogenic bacterial cellulose of the present disclosure includes cellulose fibers that act as guides that promote cellular mechanotransduction and facilitate the scaffolding of new tissues. It should be noted that the bacterial cellulose product which is currently available on the market does not meet this feature due to the alteration of its 3D structure during its production process, as described in U.S. Pat. No. 7,374,775. As mentioned before, this currently available bacterial cellulose is not used as soft tissue replacement.

### Permanence, Volume, and Accessibility

In some implementations, the non-pyrogenic bacterial cellulose of the present disclosure is characterized by an improved permanence, volume and accessibility.

Most aesthetic injections (lips, nose, jaw, etc.) that add volume are currently temporary in nature and require recurrent soft tissue replacement injections. Other surgical procedures such as in the context of an accident, disease, or genetic malformation require permanent soft tissue replacements. Increasing the lifespan of an implant could thus constitute a key factor for broadening the spectrum of applicability in surgical procedures.

Although soft tissue replacement materials such as polymethyl methacrylate (PMMA), expanded polytetrafluoroethylene (ePTFE), and silicone may be considered, they often show difficulty in integration which could lead to discomfort associated with implant encapsulation.

Under such circumstances, the non-pyrogenic bacterial cellulose of the present disclosure has the potential to offer surgeons a permanent, biocompatible option from an aesthetic perspective that can add volume when integrated into the dermis.

Furthermore, other soft tissue implants such as autologous fat need access to a fat source from another part of the body so that the recovered fat (by a process such as liposuction) can be implanted on another part of the body. This type of intervention has a high success rate but requires the patient to have enough fat for the operation. In cases of cancer, diabetes, hyperthyroidism, or infections, patients may not have enough fat for an autograft, limiting the options to those mentioned previously. However, non-pyrogenic bacterial cellulose in accordance with the present disclosure may advantageously addresses this accessibility gap.

The non-pyrogenic bacterial cellulose of the present disclosure thus offers surgeons with a viable option: a material that can provide permanent aesthetic volume that is biocompatible and integrates into the dermis—likely representing an improvement over existing solutions for aesthetic injections and an advantageous alternative for permanent soft tissue replacements.

The present inventors believe that reconstructive and plastic surgeons would find the non-pyrogenic bacterial cellulose of the present disclosure particularly valuable due to its biocompatibility, minimal inflammation, and seamless integration into the body. These properties make it an excellent choice for soft tissue replacement, offering high hydration for tissue regeneration and providing a permanent volume. This versatility is especially beneficial for various reconstructive procedures, including facial reconstruction and post-mastectomy breast reconstruction. By utilizing a reliable and adaptable solution, surgeons can reduce the need for multiple types of implants, streamlining their operations and improving patient outcomes. Further, the variability possible in terms of thickness is also a clear advantage, which allows the product to also be used as a paste-type soft tissue replacement that could provide structural filling, giving surgeons a product that can be better molded to specific defects.

The present inventors also believe that dermatologists and cosmetic surgeons would also be highly interested in the non-pyrogenic bacterial cellulose of the present disclosure for its potential applications in aesthetic enhancements as it represents a long-lasting and natural-looking alternative to existing fillers. This is particularly appealing for cosmetic enhancements such as fillers for lips, chin, and jawline. The bacterial cellulose membrane of the present disclosure low cost and non-pyrogenic nature, combined with its ability to be implanted with minimal inflammation, make it a highly attractive option for both cosmetic surgeons and their patients.

The present inventors also believe that patients undergoing reconstructive surgery would greatly benefit from the non-pyrogenic bacterial cellulose of the present disclosure. Those requiring tissue reconstruction due to injury, trauma, surgery (such as mastectomy), or congenital conditions would find the herein described properties of high hydration, biocompatibility, and permanent volume of the non-pyrogenic bacterial cellulose membrane as offering a stable and long-lasting solution. Further, the reduced risk of inflammation and high integration with native tissues ensure better outcomes and lower complication rates, making it a preferred choice for many patients in need of reconstructive procedures.

The present inventors also believe that on the cosmetic side, individuals seeking aesthetic improvements would also be interested in the non-pyrogenic bacterial cellulose of the present disclosure. Patients looking for volume enhancements in lips, cheeks, and other facial features would appreciate the non-pyrogenic bacterial cellulose of the present disclosure as a non-surgical, long-lasting option for cosmetic enhancements. The ability to achieve natural-looking results with less risk compared to other fillers that might encapsulate, or rupture makes it a desirable choice for many seeking aesthetic improvements. Minimal inflammation and excellent integration further enhance the appeal, ensuring that patients can achieve their desired look with confidence. It is also worth noting that the non-pyrogenic bacterial cellulose of the present disclosure is fully vegan, making it a very interesting option for any person that is concerned with sustainability and animal welfare.

### Remaining Affordable

In some implementations, the non-pyrogenic bacterial cellulose of the present disclosure can be produced with an affordable method.

As discussed previously, tissue replacement materials should have the following basic properties: no signs of antigenicity, lack of toxicity, preventing contracture and provoking the lowest possible inflammatory response, sterility and bacterial barrier or antibacterial properties, appropriate water volume and appropriate mechanical properties, depending on the type of reconstructed tissue, facilitating angiogenesis. In addition, it is good if they are readily available, packaged and handled, and the production and storage costs are low. It is not easy to implement because many of these features can be mutually exclusive, e.g., mechanical strength with adequate flexibility and maximum reduction of material thickness.

Advantageously, the non-pyrogenic bacterial cellulose according to the present disclosure is produced by an efficient low-cost method that can potentially meet a lower price point when compared to other products available in the soft tissue implant market, while being characterized with a combination of the desired basic properties for tissue replacement material (i.e., medical implant). For example, the herein described non-pyrogenic bacterial cellulose provides a competitive advantage in the cosmetic market. The unique properties of the bacterial cellulose, including minimal inflammation and excellent tissue integration, would appeal to both clinicians and patients, potentially leading to a higher adoption rate and significant market penetration.

In view of the foregoing, the reader will understand that the non-pyrogenic bacterial cellulose of the present disclosure represents a versatile soft tissue replacement applicable to a wide range of clinical applications. The alternatives available on the market all have shortcomings that limit their applicability to only a few medical conditions at a time.

The non-pyrogenic bacterial cellulose of the present disclosure and a method of producing same will now be described in further details.

### Bacterial Cellulose

In a broad aspect, the present disclosure relates to a versatile biomaterial composed of bacterial cellulose which is non-pyrogenic. This bacterial cellulose can be useful as bio-integrable matrices for human tissue substitutes, tissue closure reinforcement, suture buttressing, guided tissue regeneration, musculoskeletal applications, active agent delivery and tissue engineering scaffolds. In particular, the bacterial cellulose of the present disclosure can be used as a wound dressing, an implantable bulking agent instead of collagen for various types of surgeries including urological and aesthetic applications, a carrier for a biologically active agent or drug to form an implantable drug delivery device or prolonged delivery system, a gel formed with a physiologically-acceptable liquid, a gel or liquid for ophthalmic solutions and applications, a gel or fluid for skin augmentation and other cosmetic applications, bone filler, fitted sheath, implant for restoration of skeletal defects and other bone applications, tissue substitute, a surgical augmentation device such as bladder neck suspension sling, a fitted sheath for articulation of various types of prostheses such as hip and knee, a ligament or tendon scaffold for new tissue formation, a breast implant or breast reconstructive device, among others.

In particular, the bacterial cellulose of the present disclosure may be used for drug delivery, tissue engineering, soft tissue reconstruction or replacement, or regenerative medicine. More particularly, the bacterial cellulose of the present disclosure can be useful as a soft tissue implant.

In some implementations, the non-pyrogenic bacterial cellulose is in the form of a membrane. The term “membrane” is intended to include generally planar-shaped formats, such as films, foams, pellicles, layers and combinations thereof.

In some implementations, the non-pyrogenic bacterial cellulose of the present disclosure is in the form of a hydrogel. For example, it can include a highly branched fibrous network of cellulose stabilized into a heterogeneous hydrogel form.

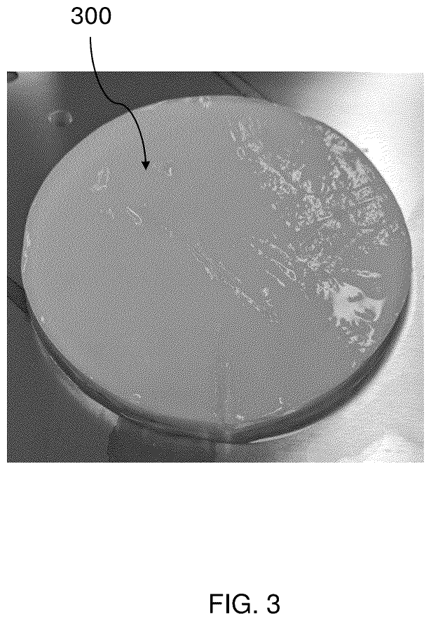

In some implementations, the non-pyrogenic bacterial cellulose of the present disclosure can take the form of membrane 300, as shown in FIG. 3. Such membrane 300 can be a flexible membrane with variable thickness and variable size. Non-limiting examples of sizes for membrane 300 may include a thickness of from about 2 mm to about 20 mm. The membrane 300 may have any length, such as from about 10 mm to about 300 mm. For example, the membrane 300 can be cut with scissors or scalpel by a user to adapt to the necessary manipulations for each surgery.

In some implementations, the non-pyrogenic bacterial cellulose of the present disclosure can be packaged and distributed in sterile, non-pyrogenic, and hydrated pouches. The pouches can contain a physiologically compatible aqueous solution. Non-limiting examples of a physiologically compatible aqueous solution includes saline solution (such as isotonic NaCl solutions).

For example, a user of the non-pyrogenic bacterial cellulose of the present disclosure can be a general surgeon, a plastic surgeon, a maxillofacial surgeon, and the like, for reconstruction or aesthetic enhancement operations requiring a bulking agent that provides permanent, durable, biocompatible, non-inflammatory, and long-lasting volume.

In some implementations, the bacterial cellulose of the present disclosure is characterized as having less than 0.5 EU/mL of endotoxins where this endotoxin level represents the true levels as it is measured with a Limulus amebocyte lysate (LAL) test on a sample of the bacterial cellulose which has been enzymatically degraded. Preferably, the bacterial cellulose of the present disclosure is characterized as having less than 0.4 EU/mL of endotoxins as measured with the LAL test, more preferably less than 0.3 EU/mL, even more preferably less than 0.2 EU/, even yet more preferably less than 0.1 EU/mL.

In some implementations, the bacterial cellulose of the present disclosure is characterized with a highly hydrated hydrogel state.

For example, the highly hydrated hydrogel state can be characterized by a water holding capacity of at least 100 g water/g dry bacterial cellulose, such as at least 150 g water/g dry bacterial cellulose, at least 200 g water/g dry bacterial cellulose, at least 250 g water/g dry bacterial cellulose, or at least 300 g water/g dry bacterial cellulose. For example, the non-pyrogenic bacterial cellulose can be characterized by a water holding capacity of from about 100 g water/g dry bacterial cellulose to about 325 g water/g dry bacterial cellulose, preferably from about 200 g water/g dry bacterial cellulose to about 325 g water/g dry bacterial cellulose.

For example, the non-pyrogenic bacterial cellulose can be characterized by a moisture content of at least about 95 wt. % moisture, such as at least about 96 wt. %, at least about 97 wt. %, at least about 98 wt. %, or at least about 99 wt. %.

For example, the non-pyrogenic bacterial cellulose can be characterized by a combination of the afore-mentioned water holding capacity and moisture content.

In some implementations, the bacterial cellulose of the present disclosure is characterized by a low percentage of fibers. For example, the bacterial cellulose membrane can include a cellulose fiber content of less than 5 wt. %, such as about 4 wt. %, about 3 wt. %, or about 2 wt. %, as determined with any suitable test method known in the art, such as with Loss-on-Drying (LOD). The present inventors have obtained non-pyrogenic bacterial cellulose with about 3 wt. % cellulose fibers. This bacterial cellulose can be in a hydrogel form. The preservation of the low percentage of fibers is also associated with high porosity, facilitating easier cell penetration into the material.

Cell penetration can be measured in average μm/day. The pore size of a tissue engineering scaffold influences cell adhesion, migration, proliferation, and differentiation. For example, it has been reported that nanopores (<100 nm) are needed for the formation of collagen fibers and extracellular matrix, whereas macropores (>100 μm) are required for cell migration and neovascularization. Conventionally, synthesized bacterial cellulose sheets have been shown in different studies to comprise a very dense meshwork of bacterial cellulose fibrils with pore sizes on the order of 0.02 to 10 μm. Without being bound by any theory, the present inventors believe that porosities closer to 100% result in larger pore sizes and enhance cell mechano-transduction through the fibers.

In some implementations, the bacterial cellulose described herein is characterized with an average cell penetration of from about 7 μm/day to about 15 μm/day. For example, the average cell penetration may be about 7 μm/day, about 8 μm/day, about 9 μm/day, about 10 μm/day, about 11 μm/day, about 12 μm/day, about 13 μm/day, about 14 μm/day, or about 15 μm/day.

In some implementations, the bacterial cellulose described herein is characterized with a reduced crystallinity level (i.e., including a non-insignificant level of amorphous content) compared to commercially available bacterial cellulose, which can be observed, for example, under a scanning electron microscope (SEM) or with X-ray diffraction (XRD) methods. For example, the bacterial cellulose described herein can be characterized by a crystallinity level of about 92% to about 95% (i.e., an amorphous level of about 5% to about 8%) as detected with XRD.

This decreased crystallinity compared to commercially available bacterial cellulose leads to increased pore size and enhanced fiber flexibility, features that can be advantageous for biocompatibility and mechano-transduction of cells in contact with the bacterial cellulose or an implant containing it. Native bacterial cellulose has an average pore size of 0.02 μm to 10 μm, significantly smaller than the 100 μm or larger pore size suitable for cell penetration. However, the reduced crystallinity (e.g., a crystallinity level of about 92% to about 95%) enables cells to displace the fibers and penetrate the bacterial cellulose more effectively than in its native form.

In some implementations, the bacterial cellulose of the present disclosure is characterized as being biocompatible and having low to nonexistent fibrosis when implanted in an individual. The versatile application of the bacterial cellulose of the present disclosure in promoting cell integration and tissue regeneration makes it invaluable for developing new therapies and products.

In some embodiments, the non-pyrogenic bacterial cellulose of the present disclosure may be used as a biocompatible carrier for various drugs, growth factors, or other therapeutic agents, enhancing their delivery and efficacy. As such, the non-pyrogenic bacterial cellulose of the present disclosure may include one or more active hydrophilic substances, such as antibiotics, anti-inflammatory agents, etc. For example, the active hydrophilic substance can be colloidal silver.

### Method of Production

A non-limiting practical implementation of a method for producing the non-pyrogenic bacterial cellulose as described herein will now be described in more details. It will be apparent to the reader that variations can be made to one or more steps of this method so long as these variations do not change the characteristics of the non-pyrogenic bacterial cellulose as described herein.

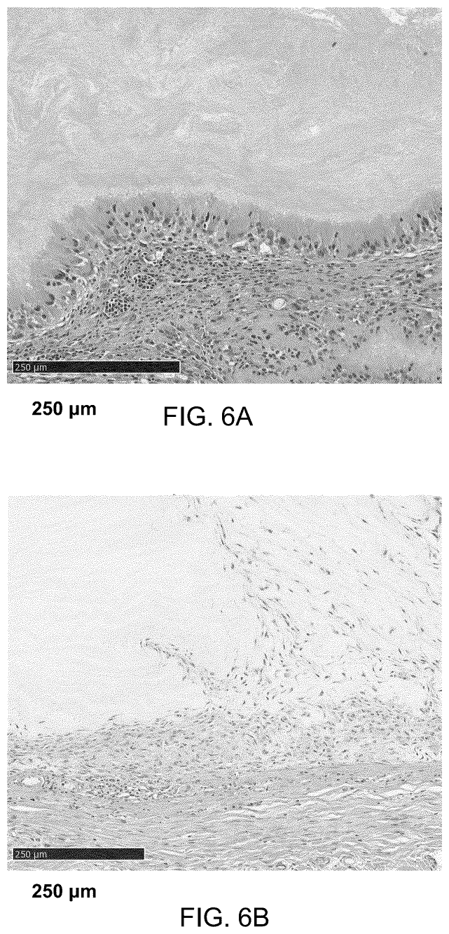

FIG. 7 is a non-limiting flowchart of method 700 for producing non-pyrogenic bacterial cellulose, in accordance with embodiments of the present disclosure.

In some implementations, the method 700 includes providing purified bacterial cellulose in an aqueous solution, step 710. This step can be performed in several ways.

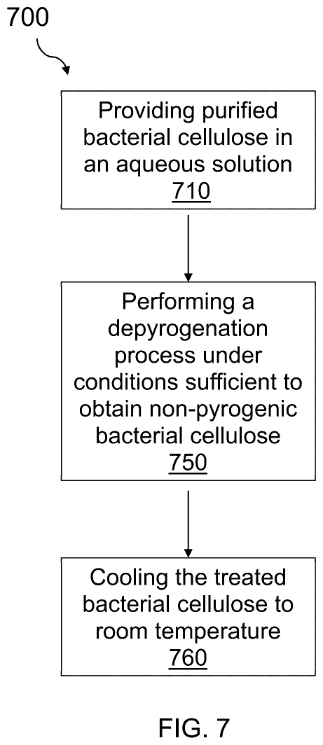

With reference to FIG. 8, the step 710 may include cultivating bacteria capable of expressing cellulose under cultivation conditions that allow expression of such cellulose, step 820. Such step can be performed in a bioreactor. The term “bioreactor” is intended to include any device or system capable of supporting a biologically active environment for growing or culturing materials. In addition to containers or vessels capable of seeding or growing bacteria, the bioreactors may also include the ability to provide agitation, pressure changes, temperature controls, humidity controls, media exchange, and ventilation. Bacterial species capable of expressing cellulose are known in the art. For example, such bacterial species can be selected amongst Gram-negative bacterium Gluconacetobacter xylinus (conventionally known as Acetobacter xylinum). This group of Gram-negative, rod-shaped aerobic bacteria, due to the high yield of cellulose, is considered to be a model organism to produce bacterial cellulose for commercial fermentation. Apart from this group, bacterial cellulose can be expressed by other bacterial genera, such as Agrobacterium spp., Acetobacter spp., Azotobacter, Rhizobium spp., Sarcina, Alcaligenes, and Pseudomonas. Acetobacter, a common vinegar bacterium, which is a non-photosynthetic advanced purple bacteria that can convert glucose, glycerol, sugar, or any other organic substances into pure cellulose. Cultivation conditions that allow expression of cellulose from Gram-negative bacteria are also known. For example, these may include inoculation in a basal nutrient broth whose main source of sugar can be glucose, and aerobic fermentation at 30° C., without agitation, allowing the formation of a superficial layer of bacterial cellulose, whose thickness increases over time. A fermentation of 2 days generally translates to 1-2 mm thick layer of bacterial cellulose.

Following the bacteria cultivation, the bacterial cellulose is isolated from the bacteria at step 830. For example, when the bacterial cellulose forms a superficial layer of bacterial cellulose (e.g., a membrane), this step may involve picking the bacterial cellulose layer with suitable means (e.g., manually). This step may also involve rinsing the bacterial cellulose with distilled water, or any other suitable aqueous solution. The bacterial cellulose can be stored at 4° C. for a period of about one week.

Alternatively, the reader will readily understand that a manufacturer may opt to forego the bacteria cultivation step 820 and instead proceed to obtain bacterial cellulose from a supplier, without personally performing the bacteria cultivation. Other possible variations are also contemplated within the scope of the present disclosure.

A purification step is then performed on the bacterial cellulose, step 840. There are several approaches to purifying bacterial cellulose.

For example, the bacterial cellulose can be purified with a cold treatment, which includes soaking the bacterial cellulose in an alkaline solution at a temperature between about 4° C. to about 40° C. for an extended period, such as 48 h. For example, the alkaline solution can include a sodium hydroxide solution, such as 0.5M sodium hydroxide. After the purification period, the alkaline solution is replaced with a buffer solution, the pH adjusted within a range from about 6 to about 8 such as about 7.4, and the bacterial cellulose is incubated in the buffer solution for an additional extended period, such as 24 h. After the additional extended period, the buffer solution is replaced with an aqueous solution, such as a saline solution (e.g., 0.9% m/v sodium chloride).

For example, the bacterial cellulose can be purified with a hot treatment, which includes soaking the bacterial cellulose in an alkaline solution at a temperature between about 50° C. and about 100° C. for a short period, such as 60 minutes. For example, the alkaline solution can include a sodium hydroxide solution, such as 0.5M sodium hydroxide. After the purification period, the alkaline solution is replaced with a buffer solution, the pH adjusted to a value of from about 6 to about 8 (e.g., about 7.4), and the bacterial cellulose is incubated in the buffer solution for an additional extended period, such as 24 h. After the additional extended period, the buffer solution is replaced with an aqueous solution, such as a saline solution (e.g., 0.9% wt./v sodium chloride).

Optionally, after these treatments, the bacterial cellulose can be autoclaved in the same aqueous solution at 121° C., 15 psi (about 0.1 MPa) for 20 minutes to obtain purified and sterile bacterial cellulose, which can be stored at 4° C. for several weeks.

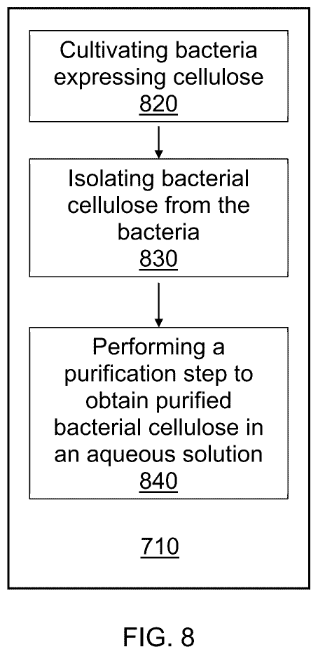

Returning to FIG. 7, the method 700 then includes performing a depyrogenation process under conditions sufficient to obtain non-pyrogenic bacterial cellulose as a hydrogel having the herein described characteristics, step 750. With reference to FIG. 9, this step may involve selecting a temperature above 140° C. and a pressure equal or higher to the pressure of saturated steam at said temperature, step 910. For example, the selected temperature for the depyrogenation process can be within the range of from about 140° C. to about 250° C., including any ranges or values therein. For example, the selected temperature for the depyrogenation process can be about 140° C., about 141° C., about 142° C., about 143° C., about 144° C., about 145° C., about 146° C., about 147° C., about 148° C., about 149° C., about 150° C., about 151° C., about 152° C., about 153° C., about 154° C., about 155° C., about 156° C., about 157° C., about 158° C., about 159° C., about 160° C., about 161° C., about 162° C., about 163° C., about 164° C., about 165° C., about 166° C., about 167° C., about 168° C., about 169° C., about 170° C., about 171° C., about 172° C., about 173° C., about 174° C., about 175° C., about 176° C., about 177° C., about 178° C., about 179° C., about 180° C., about 181° C., about 182° C., about 183° C., about 184° C., about 185° C., about 186° C., about 187° C., about 188° C., about 189° C., about 190° C., about 191° C., about 192° C., about 193° C., about 194° C., about 195° C., about 196° C., about 197° C., about 198° C., about 199° C., about 200° C., about 201° C., about 202° C., about 203° C., about 204° C., about 205° C., about 206° C., about 207° C., about 208° C., about 209° C., about 210° C., about 211° C., about 212° C., about 213° C., about 214° C., about 215° C., about 216° C., about 217° C., about 218° C., about 219° C., about 220° C., about 221° C., about 222° C., about 223° C., about 224° C., about 225° C., about 226° C., about 227° C., about 228° C., about 229° C., about 230° C., about 231° C., about 232° C., about 233° C., about 234° C., about 235° C., about 236° C., about 237° C., about 238° C., about 239° C., about 240° C., about 241° C., about 242° C., about 243° C., about 244° C., about 245° C., about 246° C., about 247° C., about 248° C., about 249° C., and about 250° C. Preferably, the selected temperature for the depyrogenation process can be within the range from about 180° C. to about 250° C., which allows a majority of bacterial cellulose to remain in the biomass solid residue, and that enables endotoxins to be decomposed at adequate rates.

The reaction pressure is preferably set to a pressure equal or higher to the pressure of saturated vapor pressure of water at the selected temperature which allows the hot compressed aqueous solution to stay liquid. For example, the reaction pressure can be set to a pressure equal to (or higher by about 0.1 MPa to about 0.5 MPa than) the saturated vapor pressure of water at each temperature, which allows the hot compressed aqueous solution to stay liquid. For example, at the selected temperatures, the selected pressure can be within the range of about 0.35 MPa to about 4.0 MPa. For example, the selected pressure can be of about 0.36 MPa at 140° C., about 1.00 MPa at 180° C., about 2.32 MPa at 220° C., and about 3.97 MPa at 250° C.

The selected temperature and pressure are then held for a period sufficient to obtain non-pyrogenic bacterial cellulose as a hydrogel having the herein described characteristics, step 920. For example, when the reaction is performed at a selected temperature of about 140° C., the selected temperature and pressure can be held for a period of about 3 h. For example, when the reaction is performed at a selected temperature of about 250° C., the selected temperature and pressure can be held for a period of about 5 minutes or more, such as 10 minutes, 20 minutes, or 30 minutes. For example, the selected period for the depyrogenation process can be within the range of from about 5 minutes to about 180 minutes, including any ranges or values therein. For example, the selected period for the depyrogenation process can be about 5 minutes, about 15 minutes, about 25 minutes, about 35 minutes, about 45 minutes, about 55 minutes, about 65 minutes, about 75 minutes, about 85 minutes, about 95 minutes, about 105 minutes, about 115 minutes, about 125 minutes, about 135 minutes, about 145 minutes, about 155 minutes, about 165 minutes, about 175 minutes, or about 180 minutes. Preferably, the selected period for the depyrogenation process can be within the range of from about 30 minutes to about 180 minutes.

The reader will readily appreciate that the herein described temperatures ranges, pressures ranges and duration ranges are only non-limiting conditions and that any conditions where the aqueous solution remains liquid over the course of treatment and affords non-pyrogenic bacterial cellulose as a hydrogel having the herein described characteristics, would also be acceptable, provided that it makes sense from an industrial implementation perspective. The present inventors have obtained best results with a reaction performed at a selected temperature of about 180° C. with a pressure of about 1.00 MPa for a period of about 3 h.

It will be appreciated by the reader that the depyrogenation process described here can be carried out in a conventional high-pressure, high-temperature hydrothermal unit capable of reaching the herein described temperatures and pressures.

Returning to FIG. 7, the method 700 may then include cooling the treated bacterial cellulose to room temperature to recover the non-pyrogenic bacterial cellulose, step 760. For example, this non-pyrogenic bacterial cellulose membrane has less than 0.5 EU/mL of endotoxins as measured with a Limulus amebocyte lysate (LAL) test of enzymatically degraded bacterial cellulose membrane, preferably less than 0.4 EU/mL of endotoxins as measured with the LAL test, more preferably less than 0.3 EU/mL of endotoxins as measured with the LAL test, even more preferably less than 0.2 EU/mL of endotoxins as measured with the LAL test, even more preferably less than 0.1 EU/mL of endotoxins as measured with the LAL test. Advantageously, the non-pyrogenic bacterial cellulose at this step of the process is characterized as having a high water content, as described above.

Optionally, the resulting non-pyrogenic bacterial cellulose membrane can be further processed, such as packaged and distributed in sterile, non-pyrogenic, and hydrated pouches.

### EXAMPLES

The following examples describe some example modes of making and practicing certain non-pyrogenic bacterial cellulose that are described herein. These examples are for illustrative purposes only and are not meant to limit the scope of the compositions and methods described herein.

### Example 1

In this example, purified native bacterial cellulose membranes are produced.

Gluconacetobacter xylinus ssp. bacteria were inoculated into a basal nutrient broth, with glucose as the primary sugar source. Aerobic fermentation took place at 30° C. without agitation, leading to the formation of a surface layer of bacterial cellulose. The membranes produced were thin, cylindrical pellets, measuring 10 mm in diameter and approximately 1.6 mm in thickness. Once formed, the membranes were manually extracted from the fermenter, rinsed with distilled water, and stored in an airtight container at 5° C. for up to one week.

A purification step was performed by soaking the bacterial cellulose membranes in a 0.5M sodium hydroxide solution at 80° C. for 60 minutes, using a ratio of 500 mL of solution per 30 g of membranes.

The sodium hydroxide solution was then discarded and replaced with a 0.1M phosphate buffer, adjusted to a pH of 7.4 with sodium hydroxide and hydrochloric acid. After 24 hours, the buffer was replaced with a 0.9% sodium chloride solution. The membranes were then autoclaved in the saline solution at 121° C. for 20 minutes. The final purified bacterial cellulose membranes were stored at 5° C. and remained stable for several weeks.

### Example 2

In this example, the purified bacterial cellulose membranes of Example 1 were processed in a depyrogenation process.

The bacterial cellulose membrane in the saline solution (0.9% m/v sodium chloride) was introduced in a high-pressure chemical autoclave. The temperature was set to a value between 180° C. and 250° C., at a pressure between 1 MPa and 4 MPa to allow the saline solution to remain liquid. The process was performed for 30 minutes at 250° C. or 3 hours at 180° C. Thereafter, the autoclave was allowed to return to room temperature before taking the treated bacterial cellulose membranes out of the system.

### Example 3

In this example, the treated bacterial cellulose membranes of Example 2 were assessed for endotoxin levels.

To access pyrogenic molecules bound to bacterial cellulose due to the polar nature of its filaments, enzymatic degradation of bacterial cellulose into simple glucose monomers was conducted. The bacterial cellulose, pre-autoclaved at 121° C. for 20 minutes, was submerged in a sterile, non-pyrogenic acetate buffer solution (0.1M, pH 4.8) at a ratio of 1 g of cellulose per 10 mL of buffer. Cellulase (C2730, Sigma-Aldrich) was added at a concentration of 1 μL/mL, and the mixture was homogenized via vortexing before being transferred to a thermostatic bath at 55° C. for 72 hours. The samples were visually inspected to ensure complete degradation of the bacterial cellulose membranes. Degraded samples were then stored at 5° C. until further use.

To quantify lipopolysaccharide (LPS) content in selected degraded samples, a Limulus amebocyte lysate (LAL) assay was used. The pH was adjusted to pH 6-8 with pyrogen-free sodium hydroxide and hydrochloric acid solutions, and the standard LAL test was conducted on the resulting solution. For example, the reader may use the Thermo Scientific™ Pierce™ LAL Chromogenic Endotoxin Quantitation Kit 88282 (Rev B.0).

A depyrogenation treatment at 180° C. for 3 hours under pressure sufficient to keep the solution liquid resulted in a bacterial cellulose membrane with an LPS content of 0.10±0.01 EU/mL. Similarly, by keeping the water at its saturated vapor pressure, depyrogenation at 250° C. for 30 minutes yielded an LPS content of 0.24±0.04 EU/mL. For comparison, a hot-purified membrane was used as a control. The results showed that the LPS concentration in the hot-purified membrane exceeded the highest detection limit by several orders of magnitude.

### Example 4

In this example, native bacterial cellulose is used as a wound dressing to assess the impact of pyrogenic molecules on wound healing.

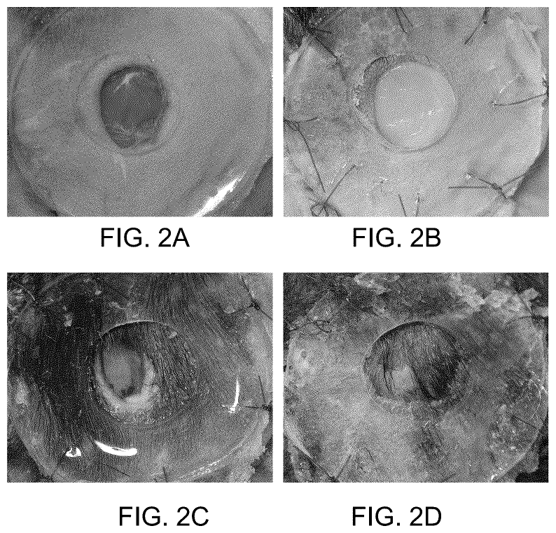

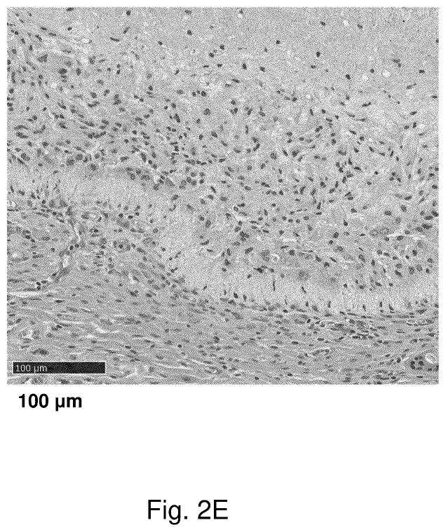

Native bacterial cellulose membranes were prepared as in Example 1. A dermal incision was made using a round biopsy dermal puncher while the rat was anesthetized. Round dressings with a hole in the center were sutured in place to provide a safe healing environment while preventing mechanical closure of the wounds. The native bacterial cellulose membrane was then applied as wound dressing and changed every day. FIGS. 2A-2D are pictures appearing in chronological order (day 0, day 3, day 10, day 14 respectively). It can be observed that the wound fully heals after 14 days without any excessive signs of redness or inflammatory reaction.

### Example 5

In this example, native bacterial cellulose is used as an implant to assess the presence of pyrogenic molecules.

Bacterial cellulose was prepared according to Example 1 and purified in hot sodium hydroxide as described above. The back of healthy rats was shaved and, while under general anesthesia, 4 subdermal pockets were made in a square pattern around the lower dorsal area. The incisions were 10 mm in width and length. The subdermal pockets were implanted with either bacterial cellulose, medical-grade silicone, or closed without any implants. All the openings were closed with nonabsorbable sutures. FIG. 2E shows a cross-section of a native bacterial cellulose membrane implanted in a healthy rat for a period of 12 weeks. The implant is situated in the bottom left region while the rat's cells are located towards the center and upper right part of the picture. There is a thick encapsulation band with macrophages and lymphocytes surrounding the implant, a sign of moderate to high inflammation.

### Example 6

In this example, a scanning electronic microscope (SEM) was used to analyze the structure of a pyrogenic bacterial cellulose membrane and of a non-pyrogenic bacterial cellulose membrane from Example 2.

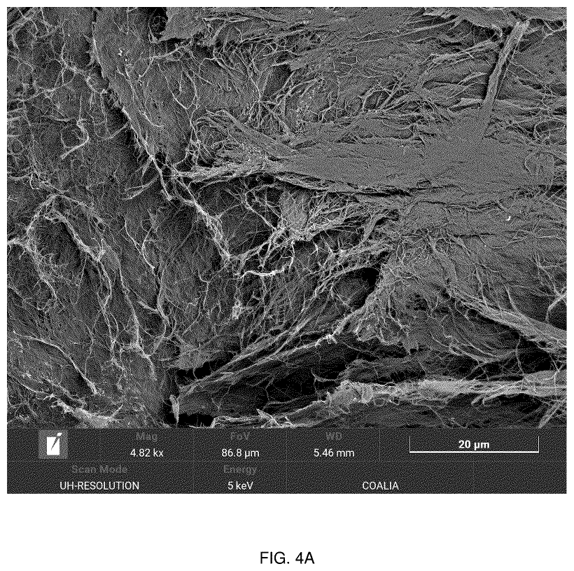

A Hitachi S-4700 FE-SEM was used with the following parameters to obtain a zoomed image of bacterial cellulose membranes: 5 keV, 4.82 kx, 86.8 μm FoV, and 5.46 mm WD (FIG. 4A) and 2 keV, 5.00 kx, 83.8 μm FoV, and 4.81 WD (FIG. 4B).

FIG. 4A shows the SEM image of the pyrogenic bacterial cellulose whereas FIG. 4B shows the SEM image of the non-pyrogenic bacterial cellulose. As can be appreciated from the two images, the non-pyrogenic bacterial cellulose membrane shows a different 3D structure characterized by lower crystallinity and larger pore size.

### Example 7

In this example, biocompatibility of the non-pyrogenic bacterial cellulose membrane used as an implant is tested in rats and compared to a medical grade silicone implant.

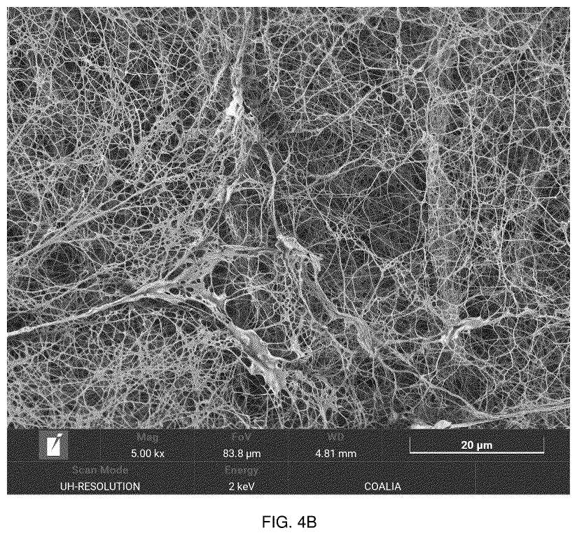

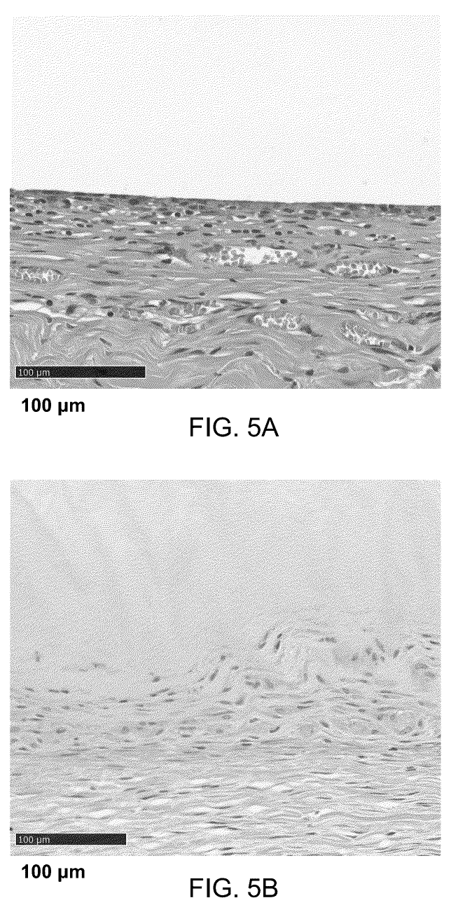

The non-pyrogenic bacterial cellulose membrane from Example 2 or a medical grade silicone implant were implanted in a rat's hypodermis the same way as described in Example 5. FIG. 5A is a histological image of the medical grade silicone implant whereas FIG. 5B is a histological image of the non-pyrogenic bacterial cellulose membrane, after only 2 weeks of implantation in the rat's hypodermis.

FIG. 5A shows evidence of moderately developed fibrosis along the implant's profile after 12 weeks. FIG. 5B shows minimal signs of inflammation and the bacterial cellulose membrane is penetrated by newly integrated myofibroblasts. The cells arranged in this pattern are well structured. The thickness of this band is between 25 and 100 μm deep. It is also worth noting that no signs of fibrosis or encapsulation bands could be found on any of the explants, suggesting that the bacterial cellulose is integrated into the rat hypodermis.

### Example 8

In this example, biocompatibility of native bacterial cellulose membrane used as an implant is tested in rats and compared to a medical grade silicone implant.

FIG. 5C is a histological image of a native bacterial cellulose membrane, 12 weeks after implantation in a rat's dermis and FIG. 5D is a histological image of silicone grade medical grade silicone implant, 12 weeks after implantation in a rat's dermis. The native bacterial cellulose membrane was purified with the conventional hot sodium hydroxide process.

The silicone implant shows signs of early encapsulation with a fibrous layer surrounding the material. Little or no signs of inflammation were detected. The native bacterial cellulose membranes exhibited acute inflammation with significant recruitment of immune cells, i.e., high density of immune cells combining mixed macrophages and neutrophils. This demonstrates that in vivo, native bacterial cellulose membrane is highly pyrogenic.

### Example 9

In this example, biocompatibility of the non-pyrogenic bacterial cellulose membrane used as an implant in rats and compared to a native bacterial cellulose membrane.

The non-pyrogenic bacterial cellulose membrane is that one from Example 2. The native bacterial cellulose membrane was purified with the conventional cold or hot sodium hydroxide process.

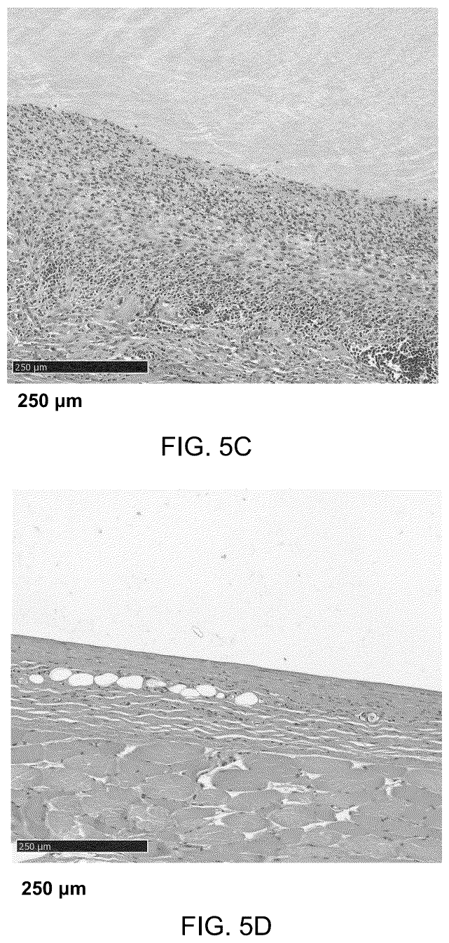

FIG. 6A is a histological image of native bacterial cellulose membrane (top of the image) implanted in a rat's dermis (bottom of the image), 12 weeks after implantation. FIG. 6B is a histological image of the non-pyrogenic bacterial cellulose membrane (top of the image) implanted in a rat's dermis (bottom of the image), 2 weeks after implantation.

FIG. 6A shows that there is a large inflammatory band with macrophages, foreign body giant cells, neutrophils, and leukocytes. FIG. 6B shows little-to-no inflammatory response, with myofibroblastic cell regeneration and a thin band (up to 100 μm) of healthy cells penetrating the non-pyrogenic bacterial cellulose membrane. Note that the FIG. 6A demonstrates that inflammatory responses persist in native and pyrogenic bacterial cellulose, even after long periods of time.

### Example 10

In this example, various comparative processes from the literature are used and compared to a process in accordance with the present disclosure in terms of endotoxin levels remaining in the bacterial cellulose.

1×PURIF. This process entails the following steps: (1) Treat bacterial cellulose with NaOH 0.5 M at 80° C. for 1 hour. (2) Rinse in distilled water for 15 min. (3) Neutralize with HCl. (4) Rinse in distilled water for 15 min. (5) Sterilize in an autoclave at 121° C. for 15 min.

2×PURIF. This process entails the following steps: (1) Treat bacterial cellulose with NaOH 0.5 M at 80° C. for 1 hour. (2) Rinse in distilled water for 15 min. (3) Repeat steps (1) and (2). (4) Neutralize with HCl. (5) Rinse in distilled water for 15 min. (6) Sterilize in an autoclave at 121° C. for 15 min.

3×PURIF. This process entails the following steps: (1) Treat bacterial cellulose with NaOH 0.5 M at 80° C. for 1 hour. (2) Rinse in distilled water for 15 min. (3) Repeat twice steps (1) and (2). (4) Neutralize with HCl. (5) Rinse in distilled water for 15 min. (6) Sterilize in an autoclave at 121° C. for 15 min.

4×PURIF. This process entails the following steps: (1) Treat bacterial cellulose with NaOH 0.5 M at 80° C. for 1 hour. (2) Rinse in distilled water for 15 min. (3) Repeat three times steps (1) and (2). (4) Neutralize with HCl. (5) Rinse in distilled water for 15 min. (6) Sterilize in an autoclave at 121° C. for 15 min.

NP. This process (described in more details in EP Patent No. 1,795,213) entails the following steps: (1) Treat bacterial cellulose with NaOH 2M at 75° C. for 1 hour. (2) Rinse in non-pyrogenic Milli-Q water for 15 min. (3) Treat with hydrogen peroxide 0.25% at 75° C. for 1 hour. (4) Rinse in non-pyrogenic Milli-Q water for 1 hour until neutralization. (5) Sterilize in an autoclave at 121° C. for 15 min.

PERFUSION. This process entails the following steps: (1) perfuse the bacterial cellulose with 0.2 L/s NaOH 0.5M at 21° C. (room temperature). (2) Change media every 2 days, changing 4 L every time. (3) treat over 14 days. (4) Neutralize with HCl. (5) Rinse in non-pyrogenic Milli-Q water for 15 min. (6) Sterilize in an autoclave at 121° C. for 15 min.

NDP. This process entails the following steps: (1) Treat bacterial cellulose with NaOH 0.5 M at 80° C. for 1 hour. (2) Soak in NaOH 0.5 M at room temperature for 48 hours. (3) Rinse in water for 15 min. (4) Neutralize with HCl. (5) Rinse in water for 15 min. (6) Sterilize in an autoclave at 121° C. for 15 min.

PERFUSION (EXT). Identical process as the PERFUSION 1 week, except that the remaining endotoxin levels were determined from the media per se and not from degraded bacterial cellulose.

HP 30-250. This process in accordance with the present invention entails the following steps: (1) Treat bacterial cellulose with NaOH 0.5 M at 80° C. for 1 hour. (2) Rinse in distilled water for 15 min. (3) Neutralize with HCl. (4) Rinse in distilled water for 15 min. (5) Depyrogenization in an autoclave at 250° C., at the saturated vapor pressure of water (about 4 MPa), for 30 min. (6) Rinse in non-pyrogenic Milli-Q water for 15 min.

NPMC. This process (described in more details in U.S. Pat. No. 7,374,775) entails the following steps: (1) Treat bacterial cellulose with NaOH 2M at 75° C. for 1 hour. (2) Rinse in non-pyrogenic Milli-Q water for 15 min. (3) Treat with hydrogen peroxide 0.25% at 75° C. for 1 hour. (4) Rinse in non-pyrogenic Milli-Q water for 1 hour until neutralization. (5) Infuse in methanol 100% during 2-7 days. (6) Treat with supercritical CO₂ for 3 hours at 2000 psi and 40° C. until the methanol was removed and the material dry. (7) Sterilized with gamma rays at 35 kGy.

HP 180-180. This process in accordance with the present invention entails the following steps: (1) Treat bacterial cellulose with NaOH 0.5 M at 80° C. for 1 hour. (2) Rinse in distilled water for 15 min. (3) Neutralize with HCl. (4) Rinse in distilled water for 15 min. (5) Depyrogenization in an autoclave at 180° C., at the saturated vapor pressure of water (about 1 MPa), for 3 hours (180 minutes). (6) Rinse in non-pyrogenic Milli-Q water for 15 min.

Samples were treated with each process and the endotoxin levels were determined as described in the present disclosure. The results are listed in the following Table 2.

TABLE 2

| | Assay 1 | Assay 2 | | | Assay 3 | Average |
|---|---|---|---|---|---|---|
| Process | (EU/ml) | (EU/ml) | | | (EU/ml) | (EU/ml) |
| 1x Purif | THTC | | | | | THTC |
| 2x Purif | THTC | | | | | THTC |
| 3x Purif | THTC | | | | | THTC |
| 4x Purif | THTC | | | | | THTC |
| Perfusion | | 3.96 | 4.02 | 4.35 | | 4.11 |
| HP 180-180 | | 0.09 | 0.1 | 0.11 | 0.19 | 0.12 |
| HP 30-250 | | 0.23 | 0.29 | 0.2 | 0.17 | 0.22 |
| Perfusion (ext) | | 0.61 | 0.6 | 0.53 | | 0.58 |
| NDP | | | | | 1.45 | 1.45 |
| NPMC | | | | | 0.19 | 0.19 |
| NP | | | | | 75.07 | 75.07 |

THTC: Too high to count

From these results, the reader will readily understand that treatment with NaOH for 1 hour, with a concentration of 0.5M to 2M is not sufficient to reduce the endotoxin levels to less than 0.5 EU/mL. The addition of a 0.25% hydrogen peroxide treatment for 1 hour is also not sufficient to reduce the endotoxin levels to less than 0.5 EU/mL. Even an extended perfusion time was not sufficient to reduce the endotoxin levels to less than 0.5 EU/mL.

Treatment with the NP procedure is also not sufficient to reduce the endotoxin levels to less than 0.5 EU/mL. It is only when adding the extended methanol and the supercritical CO₂ treatment (i.e., NPMC process) that the inventors were able to reduce the endotoxin levels to less than 0.5 EU/mL. Importantly, the reader will recognize that the NPMC process dehydrates the bacterial cellulose and limits the maximum rehydration levels since the resulting thickness obtained after subsequent water exposure (i.e., “swelling”) was 0.5 mm.

In contrast, bacterial cellulose obtained with a process according to the present disclosure (e.g., HP 180-180 or HP 30-250) can have high water content (at least 95 wt. %) and variable thickness, such as from about 2 mm to about 20 mm. Thus, the present inventors can produce much thicker and hydrated membranes compared to those obtainable with the NPMC process, which is advantageous for downstream medical applications such as reconstructive surgeries where much larger medical implant devices are typically required.

### Example 11

In this example, various comparative processes from the literature are used and compared to a process in accordance with the present disclosure in terms of endotoxin levels remaining in the bacterial cellulose, water content and water holding capacity.

Fermentation: The bacterial cellulose was obtained as in Example 1 except that the fermentation was conducted over 4 days to obtain 2-4 mm thick membranes. In this experiment, all the membranes used for testing were cylindrical sheets with diameters of 80 mm and thicknesses between 4 and 8 mm. Once the membranes were produced, the membranes were manually removed from their fermenter and rinsed with distilled water.

Purification: The membranes were purified by soaking in a 0.5 M sodium hydroxide solution at 80° C. for 20 min. A ratio of 500 mL sodium hydroxide solution per 30 g of membranes was used. This process was repeated twice, followed by a succession of 20 min washes with boiling deionized water. The final pH was comprised between 6 and 8. The membranes were autoclaved at 121° C. for 20 min.

Sample processing: Following purification, every membrane was cut into 6 wedges, allowing for more testing capacity and replicability. From this point, 2 cm×2 cm pieces of SYNTHECEL® Dura Repair (Batch 231004056, DePuy Synthes, Warsaw, Indiana, USA) were cut and stored separately.

Depyrogenation: Following processing, the membranes were depyrogenized in a 1 L hydrothermal reactor (236HC10 T-316, 111744, PARR Instrument Company, Moline, Illinois, USA) by adding some samples to 200 mL of deionized water. The reactor was evenly heated with a heating mantle to temperatures between 140° C. and 250° C. The treatment took between 30 minutes and 3 hours depending on the desired heat. Saturated vapor pressure was reached depending on the temperature (0.36 MPa at 140° C., 1.00 MPa at 180° C., 2.32 MPa at 220° C., and 3.97 MPa at 250° C.). The membranes were allowed to fully cool down before being removed from the reactor.

Some samples were not depyrogenized (Not DePyrogenized, NDP). Some other samples were, instead of depyrogenized, autoclaved again (120° C., 0.20 MPa) for a total of 3 hours. Finally, some samples were simply boiled for 3 h in a beaker with excess deionized water.

Oven drying: 5 cm×5 cm aluminum foil squares were manually cut, identified, then weighed using a precision electronic balance (USS-DBS83-220IC, 1022504058, U.S. Solid, Cleveland, Ohio, USA). The depyrogenized samples were gently blotted up using absorbent paper for a couple of seconds to remove excess water. The samples were individually placed on the aluminum foil squares, and their masses were measured again. The samples were then transferred to an oven preheated at 150° C. The samples were allowed to dry for 36 hours, then cooled down for about 10 minutes. The samples were weighed one last time to calculate their water content and holding capacity.

Sample degradation and endotoxin quantification: To gain access to molecules bound to bacterial cellulose or those with low extractability due to the polar nature of bacterial cellulose filaments, enzymatic bacterial cellulose degradation into simple glucose monomers was performed. Bacterial cellulose used for degradation was previously autoclaved at 121° C. for 20 minutes. Bacterial cellulose membranes were submerged in a sterile and non-pyrogenic solution of acetate buffer (0.1M, pH 4.8) at a ratio of 1 g per 10 mL of buffer. To these samples, 1 μL/mL of cellulase (C2730, Sigma-Aldrich) was added. The mixture was homogenized by vortexing and transferred to a thermostated bath at 55° C. for 72 hours. During the degradation, samples were regularly agitated to promote the enzymatic bacterial cellulose degradation. Degraded samples were visually inspected to confirm the total disappearance of the bacterial cellulose membranes. Samples were stored at 5° C. until use.

Following the digestion, a Limulus amebocyte lysate (LAL) (PI88282, Thermo Scientific) test was used to quantify LPS content in selected samples. The pH was adjusted using pyrogen-free solutions made of sodium hydroxide and hydrogen chloride. A standard LAL test was then performed on the resulting solution.

The water content (w) was calculated as follows:

$$w = 1 - \frac{{m}_{dry \_ tot} - {m}_{alu}}{{m}_{wet \_ tot} - {m}_{alu}}$$

Where m_{dry_tot} is the combined mass of the aluminum foil and dried samples, m_{wet_tot} is the combined mass of the aluminum foil and wet samples and mₐₗᵤ is the mass of the aluminum foil squares alone.

The water holding capacity (WHC) is defined as the number of times a dry product can hold its own weight in water. It is a ratio, and therefore, dimensionless. It is intuitively described as the water content divided by the dry content:

$$WHC = \frac{w}{1 - w}$$

Or, using the original variables and simplifying:

$$WHC = \frac{{m}_{dry \_ tot} - {m}_{a l u}}{{m}_{wet \_ tot} - {m}_{dry \_ tot}}$$

Statistical significance was performed using an ANOVA, followed by a Tukey HSD for pairwise comparison. All statistical computations were calculated using R version 4.5.0 (2025-04-11 ucrt) in RStudio® (Posit Software, PBC, 2025).

Results were taken as averages, and error was propagated using the partial derivative formula:

$${σ}_{F} = \sqrt{{( \frac{\partial F}{\partial x} )}^{2} {σ}_{x}^{2} + {( \frac{\partial F}{\partial y} )}^{2} {σ}_{y}^{2} + {( \frac{\partial F}{\partial z} )}^{2} {σ}_{z}^{2} + …}$$

The average of all σ_{F}_{ᵢ} was then taken to get the mean systematic error of the balance for every condition.

$${σ}_{sys , avg , balance} = {\sum }_{i = 1}^{n} \frac{{σ}_{{F}_{i}}}{n}$$

For randomized errors, the standard error formula was used. This uses the standard deviation as a parameter.

$${σ}_{rand} = \frac{σ}{\sqrt{n}}$$

The errors were then combined using the quadrature sum of all sources of error:

$${σ}_{tot} = \sqrt{{σ}_{sys , avg , balance}^{2} + {σ}_{rand}^{2}}$$

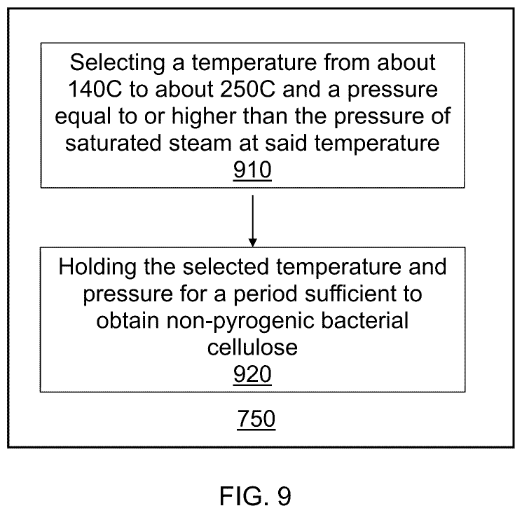

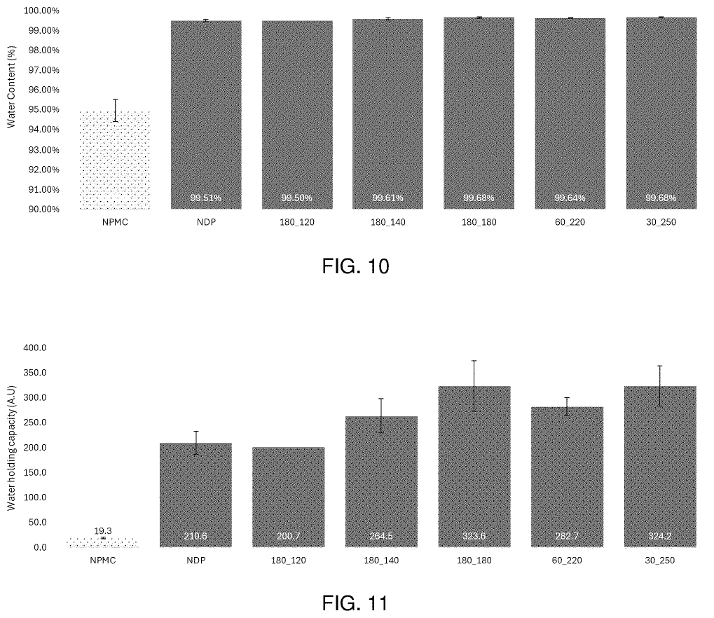

Results are reported in the following Table 3 and in FIG. 10 and FIG. 11.

TABLE 3

| | No. of | | | | | | |
|---|---|---|---|---|---|---|---|
| | Samples | | | | | | |
| | (for water | | | | | Number of | |
| | content | | | Water | | Samples | Endotoxin |
| | & holding | Water | Total | holding | Total | (for | Content |
| | capacity) | content | error | capacity | Error | endotoxin) | (EU/mL) |
| NPMC | 3 | 94.98% | 0.57% | 19.3 | 2.0 | 3 | <0.1 |
| NDP | 3 | 99.51% | 0.06% | 210.6 | 23.1 | 3 | >0.5* |
| 180_100 | 1 | N/A | | N/A | | 3 | >0.5* |
| 180_120 | 1 | 99.50% | | 200.7 | | 3 | >0.5* |
| 180_140 | 3 | 99.61% | 0.06% | 264.5 | 35.1 | 1 | <0.1 |
| 180_180 | 3 | 99.68% | 0.05% | 323.6 | 50.9 | 3 | <0.1 |
| 60_220 | 3 | 99.64% | 0.03% | 282.7 | 18.2 | 3 | <0.1 |
| 30_250 | 3 | 99.68% | 0.04% | 324.2 | 41.0 | 3 | <0.1 |

*Results higher than 0.5 EU/mL vary greatly because they depend on the endotoxin concentration in the original sample. Therefore, they are only reported as >0.5.

Results show that native bacterial cellulose (NDP) has a water content of 99.51%±0.06% (WHC of 210.6±23.1 g water/g dry bacterial cellulose), which aligns with previous reports on bacterial cellulose (R Rebelo A et al., Dehydration of bacterial cellulose and the water content effects on its viscoelastic and electrochemical properties. Sci Technol Adv Mater. 2018 Mar. 9; 19(1):203-211. doi: 10.1080/14686996.2018.1430981. PMID: 29707063; PMCID: PMC5917443). All the samples that were either boiled or depyrogenized in a hydrothermal reactor maintained their water content, and by association, their water holding capacity.

FIG. 10 and FIG. 11 show that the NPMC samples are significantly less hydrated than native bacterial cellulose, both in water content and water holding capacity (p<0.0001 for water content and p<0.05 for water holding capacity) with NPMC showing a 94.98% water content equivalent to a 10.9-fold reduction in water holding capacity (from 210.6 to 19.3). When excluding the NPMC samples, all statistical pairwise comparison showed no significant distinction between groups, again suggesting that water holding capacity and water content is preserved when hydrothermal depyrogenation is performed, regardless of temperature, pressure and time.

In this experiment, it is shown that the cutoff temperature point should hover around 140° C. The present inventors do not expect that temperatures lower than 140° C. could produce endotoxin-free samples within acceptable timeframes for industrial applications. For higher temperatures, all samples were successfully depyrogenized within an acceptable timeframe.

### Example 12

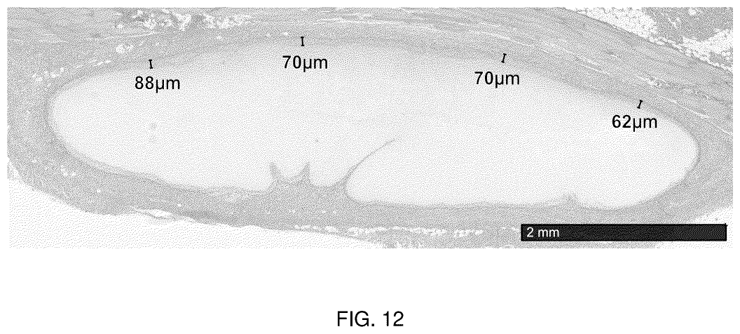

In this example, the cell penetration values in a bacterial cellulose membrane of the present disclosure are taken at 4 evenly distributed locations on the implants as shown in FIG. 12. The bacterial cellulose was processed at 180° C. for 180 minutes under the depyrogenization conditions of the invention, as described in the previous examples. The software used for analyzing the results was NDP.view2.

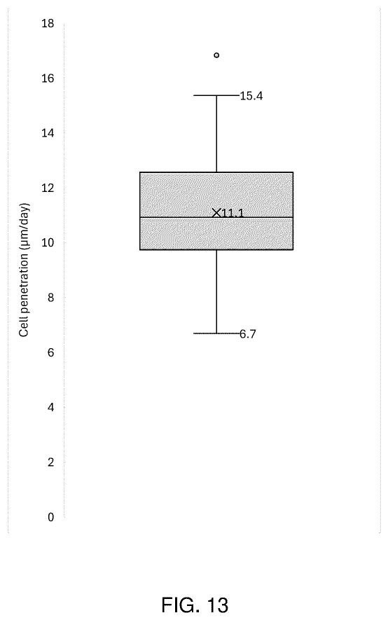

Results of cell penetration are reported in the following table and in FIG. 13.

TABLE 4

| | | | | | | | Average |
|---|---|---|---|---|---|---|---|
| | | Center | Center | | Average | Implantation | Migration speed |
| Sample | Left | left | right | Right | penetration | time (day) | (μm/day) |
| 1PD | 51 | 85 | 123 | 136 | 98.75 | 14 | 7.053571429 |
| 1TD | 137 | 188 | 188 | 123 | 159 | 14 | 11.35714286 |
| 2PD | 97 | 82 | 92 | 166 | 109.25 | 14 | 7.803571429 |
| 2TD | 177 | 64 | 291 | 125 | 164.25 | 14 | 11.73214286 |
| 3PD | 152 | 172 | 218 | 117 | 164.75 | 14 | 11.76785714 |
| 3TD | 133 | 131 | 150 | 274 | 172 | 14 | 12.28571429 |
| 4PD | 142 | 197 | 67 | 136 | 135.5 | 14 | 9.678571429 |
| 4TD | 64 | 99 | 123 | 88 | 93.5 | 14 | 6.678571429 |
| 5PD | 78 | 90 | 112 | 89 | 92.25 | 7 | 13.17857143 |
| 5TD | 60 | 136 | 116 | 118 | 107.5 | 7 | 15.35714286 |
| 6PD | 66 | 134 | 160 | 111 | 117.75 | 7 | 16.82142857 |
| 6TD | 54 | 131 | 46 | 46 | 69.25 | 7 | 9.892857143 |
| 7PD | 48 | 109 | 92 | 44 | 73.25 | 7 | 10.46428571 |
| 7TD | 55 | 59 | 86 | 154 | 88.5 | 7 | 12.64285714 |
| 8PD | 48 | 80 | 81 | 76.3 | 71.325 | 7 | 10.18928571 |
| 8TD | 88 | 70 | 70 | 62 | 72.5 | 7 | 10.35714286 |

The average migration speed was 11.07879464 μm/day with a standard deviation of 2.731561709 μm/day.

### REFERENCES

[1] A. M. Weiss, N. Macke, Y. Zhang, C. Calvino, A. P. Esser-Kahn, et S. J. Rowan, In Vitro and in Vivo Analyses of the Effects of Source, Length, and Charge on the Cytotoxicity and Immunocompatibility of Cellulose Nanocrystals, ACS Biomater. Sci. Eng., vol. 7, no 4, p. 1450-1461, April 2021, doi: 10.1021/acsbiomaterials.0c01618.
[2] M. A. Pigaleva et al., A new approach to purification of bacterial cellulose membranes: What happens to bacteria in supercritical media?, J. Supercrit. Fluids, vol. 147, p. 59-69, May 2019, doi: 10.1016/j.supflu.2019.02.009.
[3] Héctor Martinez Ávila et al., Biocompatibility evaluation of densified bacterial nanocellulose hydrogel as an implant material for auricular cartilage regeneration, Appl. Microbiol. Biotechnol., 2014, doi: 10.1007/s00253-014-5819-z.
[4] Hector Martinez Avila et al., Novel bilayer bacterial nanocellulose scaffold supports neocartilage formation in vitro and in vivo., Biomaterials, 2015, doi: 10.1016/j.biomaterials.2014.12.025.
[5] A. Bodin, S. Bharadwaj, S. Wu, P. Gatenholm, A. Atala, et Y. Zhang, Tissue-engineered conduit using urine-derived stem cells seeded bacterial cellulose polymer in urinary reconstruction and diversion, Biomaterials, vol. 31, no 34, p. 8889-8901, December 2010, doi: 10.1016/j.biomaterials.2010.07.108.
[6] J. J. Harris, G. Serafica, C. J. Damien, et H. R. Nonnenmann, Oxidized microbial cellulose and use thereof, CA2645764C.
[7] G. Serafica, R. Mormino, G. A. Oster, K. E. Lentz, et K. P. Koehler, Microbial cellulose wound dressing comprising phmb, CA2524184C.
[8] C. J. Damien, H. A. Beam, G. A. Oster, F. S. Wright, et G. Serafica, Dura substitute and a process for producing the same, CA2536523C.
[9] R. M. B. Jr, W. Czaja, M. Jeschke, et D. J. Young, Multiribbon nanocellulose as a matrix for wound healing, U.S. Pat. No. 8,951,551B2.
[10] H. A. Beam, C. J. Damien, et G. A. Oster, Thermally modified microbial-derived cellulose for in vivo implantation, AU2004266155B2.
[11] G. Serafica, R. Mormino, G. A. Oster, K. E. Lentz, et K. P. Koehler, Microbial cellulose wound dressing for treating chronic wounds, U.S. Pat. No. 7,704,523B2.

All references cited throughout the specification are hereby incorporated by reference in their entirety for all purposes.

Note that titles or subtitles may be used throughout the present disclosure for convenience of a reader, but in no way these should limit the scope of the invention. Moreover, certain theories may be proposed and disclosed herein; however, in no way they, whether they are right or wrong, should limit the scope of the invention so long as the invention is practiced according to the present disclosure without regard for any particular theory or scheme of action.

As used herein, the wording “independently selected” in reference to a group of specified items refers to the fact that when more than one item is selected from the group of items, the decision of selecting a specific item is not influenced by the decision of selecting any of the previous or following item(s).

Reference throughout the specification to “some embodiments”, and so forth, means that a particular element (e.g., feature, structure, and/or characteristic) described in connection with the invention is included in at least one embodiment described herein, and may or may not be present in other embodiments. In addition, it is to be understood that the described inventive features may be combined in any suitable manner in the various embodiments.

It will be understood by those of skill in the art that throughout the present specification, the term “a” used before a term encompasses embodiments containing one or more to what the term refers. It will also be understood by those of skill in the art that throughout the present specification, the term “comprising”, which is synonymous with “including,” “containing,” or “characterized by,” is inclusive or open-ended and does not exclude additional, un-recited elements or method steps.

Unless otherwise defined, all technical and scientific terms used herein have the same meaning as commonly understood by one of ordinary skill in the art to which this invention pertains. In the case of conflict, the present document, including definitions will control.

As used in the present disclosure, when the terms “around”, “about” or “approximately” are before a quantitative value, the present disclosure also includes the specific quantitative value itself, unless specifically stated otherwise. As used herein, the terms “around”, “about” or “approximately” refer to a ±10% variation from the nominal value unless otherwise indicated or inferred.

Unless otherwise noted, the expression “at least” or “at least one of” as used herein includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Unless otherwise noted, the order of steps or order for performing certain actions is immaterial so long as the present invention remain operable. Moreover, two or more steps or actions may be conducted simultaneously.

Unless otherwise noted, the use of any and all examples, or exemplary language herein, for example, “such as” or “including,” is intended merely to illustrate better the present invention and does not pose a limitation on the scope of the invention. No language in the specification should be construed as indicating any non-claimed element as essential to the practice of the present invention.

Although various embodiments of the disclosure have been described and illustrated, it will be apparent to those skilled in the art considering the present description that numerous modifications and variations can be made. The scope of the invention is defined more particularly in the appended claims.

1. Non-pyrogenic bacterial cellulose characterized as being a hydrogel having a water holding capacity of at least 100 g water/g dry bacterial cellulose and having less than 0.5 EU/mL of endotoxins, measured with a Limulus amebocyte lysate test on a sample of the bacterial cellulose membrane which has been enzymatically degraded.

2. The bacterial cellulose of claim 1, wherein the water holding capacity is of at least 150 g water/g dry bacterial cellulose, at least 200 g water/g dry bacterial cellulose, at least 250 g water/g dry bacterial cellulose, or at least 300 g water/g dry bacterial cellulose.

3. The bacterial cellulose of claim 1, comprising a water content of at least 95 wt. %, at least 96 wt. %, at least 97 wt. %, or at least 98 wt. %, or at least 99 wt. %.

4. The bacterial cellulose of claim 1, wherein the hydrogel is in a membrane form.

5. The bacterial cellulose of claim 4, wherein the membrane has a thickness of at least 2 mm.

6. The bacterial cellulose of claim 1, characterized with an average cell penetration of from about 7 μm/day to about 15 μm/day.

7. The bacterial cellulose of claim 1, comprising a crystallinity level of about 92% to about 95%.

8. The bacterial cellulose of claim 1, comprising pores having a size facilitating cell migration and neovascularization.

9. (canceled)

10. A manufacturing method, comprising:
a) providing a purified bacterial cellulose in an aqueous solution;
b) performing a depyrogenation process under conditions sufficient to obtain a non-pyrogenic bacterial cellulose having an endotoxin level of less than 0.5 EU/mL measured with a Limulus amebocyte lysate test on a sample of the non-pyrogenic bacterial cellulose which has been enzymatically degraded,
wherein the conditions include a process temperature within the range of from about 140° C. to about 250° C. and a pressure which allows the aqueous solution to stay liquid.

11. The method of claim 10, wherein the pressure is equal to the saturated vapor pressure of water at the process temperature.

12. The method of claim 10, wherein the process temperature is from about 180° C. to about 250° C.

13. The method of claim 10, wherein the pressure is within the range of from about 0.35 MPa to about 4 MPa.

14. The method of claim 10, wherein the depyrogenation process is performed for a period of from about 30 minutes to about 3 hours.

15. The method of claim 10, wherein the depyrogenation process is performed in a high-pressure, high-temperature hydrothermal reactor unit.

16. The method of claim 10, further comprising cooling the non-pyrogenic bacterial cellulose to room temperature.

17. The method of claim 10, further comprising packaging the non-pyrogenic bacterial cellulose in a sterile, non-pyrogenic, and hydrated pouch.

18. The method of claim 10, wherein the aqueous solution is isotonic saline solution.

19. (canceled)

20. (canceled)

21. The method of claim 10, wherein the non-pyrogenic bacterial cellulose is characterized as being a hydrogel having a water holding capacity of at least 100 g water/g dry bacterial cellulose.

22. The method of claim 21, wherein the purified bacterial cellulose and the non-pyrogenic bacterial cellulose are in a membrane form.
